(19) United States
(12) Patent Application Publication
(10) Pub. No.: US 2021/0350674 A1
(43) Pub. Date: Nov. 11, 2021

(54) SANITIZING ELECTRONIC GAMING MACHINES AND VIRUS PREVENTION IN GAMING ENVIRONMENT

(71) Applicant: IGT, Las Vegas, NV (US)

(72) Inventors: Dwayne R. Nelson, Las Vegas, NV (US); Kevin Higgins, Reno, NV (US); Erik Petersen, Sparks, NV (US)

(21) Appl. No.: 17/307,272

(22) Filed: May 4, 2021

Related U.S. Application Data
(60) Provisional application No. 63/020,130, filed on May 5, 2020.

(51) Int. Cl. G07F 17/32; A61L 2/10; A61L 2/24; A61L 2/28

(52) U.S. Cl. CPC G07F 17/3269; G07F 17/3206; G07F 17/3232; G07F 17/34; A61L 2/10; A61L 2/24; A61L 2/28; G07F 17/3211

(57) ABSTRACT

Various systems, apparatus, and methods for sanitizing electronic gaming machines and microorganisms prevention in a gaming environment.

### PRIORITY

This application claims priority to and the benefit of U.S. Provisional Patent Application No. 63/020,130, filed May 5, 2020, the entire contents of which is incorporated herein by reference.

### BACKGROUND

Electronic gaming machines (“EGMs”) refer to any suitable electronic gaming machine that enables a player to play one or more games, wherein the EGM comprises, but is not limited to: a slot machine, a video poker machine, a video lottery terminal, a terminal associated with an electronic table game, a video keno machine, a video bingo machine located on a casino floor, a sports betting terminal, or a kiosk (such as but not limited to a sports betting kiosk). As players interact with EGMs, they may transmit microorganisms such as bacteria and viruses to the EGM surfaces that could potentially spread to other players.

### BRIEF SUMMARY

Various embodiments of the present disclosure provide a casino management system including a processor and a memory device that stores a plurality of instructions, which when executed by the processor, cause the processor to: (a) responsive to receiving a player departing event signal representing an indication that a player has left an EGM, cause a sanitization requirement notification associated with the EGM to be provided to a casino staff member; and (b) responsive to receiving a sanitization complete event signal representing an indication that the EGM has been sanitized, cause a state of the EGM to indicate that the EGM has been sanitized.

Various embodiments of the present disclosure provide an EGM including a display device, a processor, and a memory device that stores a plurality of instructions, which when executed by the processor, cause the processor to: (a) responsive to detecting that a player has left the EGM, send a player departing event signal representing an indication that the EGM needs to be sanitized; and (b) responsive to receiving an indication that the EGM has been sanitized, display, via the display device, an indication that the EGM has been sanitized.

Various embodiments of the present disclosure provide an EGM including a display device, a processor, and a memory device that stores a plurality of instructions, which when executed by the processor, cause the processor to: (a) responsive to an occurrence of a sanitization event, cause the EGM to be in a clean state in which the EGM is not being played and has been sanitized; (b) responsive to an occurrence of a player occupying event, cause the EGM to be in an active state; and (c) responsive to an occurrence of a departing event, cause the EGM to be eligible to be in a locked state, and then cause the EGM to be in a locked state that requires an occurrence of a sanitization event before the EGM returns to the clean state.

Additional features are described herein, and will be apparent from the following Detailed Description and the figures.

### BRIEF DESCRIPTION OF SEVERAL VIEWS OF THE DRAWINGS

FIG. 1 depicts an example ultraviolet light bar that extends from the top of an EGM in accordance with one example embodiment of the present disclosure.

FIG. 2 depicts an example of a covering for an EGM that may be motorized or manually operated by a player in accordance with one example embodiment of the present disclosure.

FIG. 3 depicts a system architecture for enabling a mobile device to control an EGM in accordance with one example embodiment of the present disclosure.

FIG. 4 depicts an example connection process and illustrates on screen instructions presented to a use for establishing a connection between the mobile device and the EGM in accordance with one example embodiment of the present disclosure.

FIG. 5 depicts an example process for supporting EGMs and games with dynamic button variants in accordance with one example embodiment of the present disclosure.

FIG. 6 depicts an example process for validating a connection with a mobile device in accordance with one example embodiment of the present disclosure.

FIG. 7 depicts systems for supporting remote control of EGMs from multiple manufacturers in accordance with one example embodiment of the present disclosure.

FIG. 8 depicts an additional high-level architecture view for supporting remote control of EGMs from multiple manufacturers in accordance with one example embodiment of the present disclosure.

FIG. 9 depicts a high-level architecture where players can remotely control an EGM button panel via a mobile application in accordance with one example embodiment of the present disclosure.

FIG. 10 depicts an architecture for enabling remote control of an EGM with a traditional wired switch button panel in accordance with one example embodiment of the present disclosure.

FIG. 11 depicts an architecture for enabling remote control of an EGM with button panels which interface via USB with EGM in accordance with one example embodiment of the present disclosure.

FIG. 12 depicts an architecture for enabling remote control of an EGM through a direct connection with the EGM from the SMIB in accordance with one example embodiment of the present disclosure.

FIG. 13 depicts an architecture for enabling remote control of an EGM with a DPP button panel from third party manufacturers in accordance with one example embodiment of the present disclosure.

FIG. 14 depicts a high-level architecture view of the system for detecting adjacent EGMs to be disabled in accordance with one example embodiment of the present disclosure.

FIG. 15 illustrates one example sequence where the EGM or SMIB describes the set of adjacent EGMs in a single message in accordance with one example embodiment of the present disclosure.

FIG. 16 illustrates one example sequence diagram where the EGM or SMIB describes the set of adjacent EGMs in a message or event per adjacent EGM or SMIB in accordance with one example embodiment of the present disclosure.

FIG. 17 illustrates the background process of periodically broadcasting the EGM or SMIBs information so that it can be gathered by adjacent EGMs or SMIBs for maintaining their own adjacent EGM or SMIB list in accordance with one example embodiment of the present disclosure.

FIG. 18 depicts one example embodiment where the EGM is connected to a SMIB and the SMIB is communicating with a Casino Management System in accordance with one example embodiment of the present disclosure.

FIG. 19 describes a standalone implementation in the EGM itself that does not require the involvement of the resident Casino Management System in accordance with one example embodiment of the present disclosure.

FIG. 20 depicts example states of an EGM during a sanitization workflow in accordance with one example embodiment of the present disclosure.

FIG. 21 depicts a flowchart of a player cashing into the EGM, playing a game, and then cashing out in accordance with one example embodiment of the present disclosure.

FIG. 22 depicts different lamp modes and user interface displays for an EGM which represent that current state of the EGM in accordance with one example embodiment of the present disclosure.

### DETAILED DESCRIPTION

### Sanitizing EGMs and Other Gaming Surfaces Using Ultraviolet Light

Certain frequencies of ultraviolet (UV) light or electromagnetic radiation have proven effective for sterilization. Specifically, short-wavelength UV light, referred to as UVC, is used in UV germicidal irradiation (UVGI) devices to disinfect surfaces by destroying or inactivating microorganisms including viruses and bacteria. Other frequencies of ultraviolet, such as far ultraviolet (FUV or Far-UVC), may be utilized in UVGI devices and have proven effective at killing microorganisms while also being safe for human exposure. UVGI devices can be integrated into or attached to EGMs and otherwise utilized in a gaming environment to disinfect surfaces and provide a safe environment for patrons.

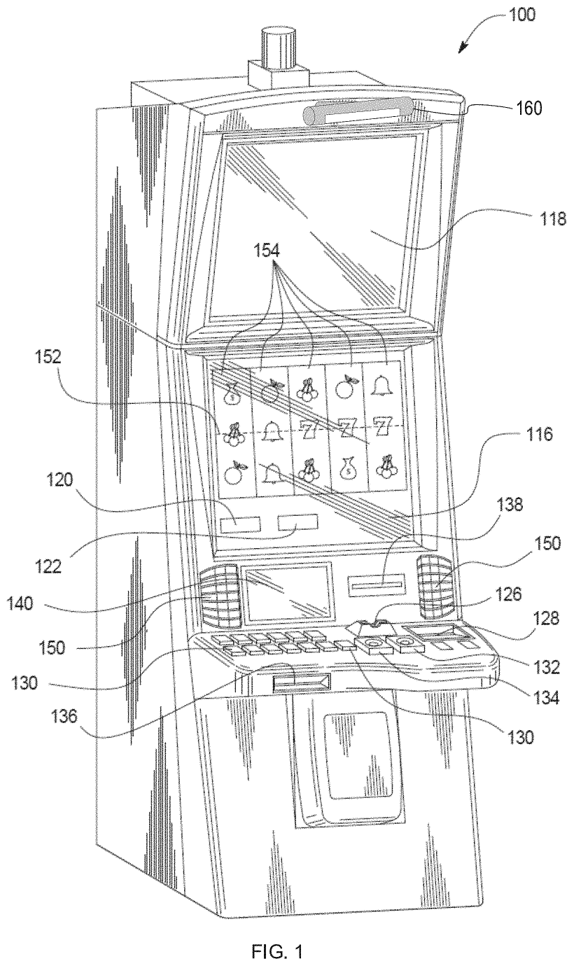

UVGI devices are available in a variety of form factors and may be integrated into an EGM in different ways in accordance with the present disclosure. FIG. 1, for instance, depicts an example UVGI light bar 160 that extends from the top of the display 118 of an EGM 100 and that shines UV light over the face of the EGM 100. The light bar 160 can be equipped with a UVC or FUV light bulb, which when activated ,disinfects the front surfaces of the EGM and surrounding air. The light bar 160 can also extend or rotate away from the EGM to shine on additional surfaces of the EGM or on a chair at the EGM in various embodiments of the present disclosure.

The size, location, and number of UVGI devices on an EGM can vary in accordance with the present disclosure. For example, the card reader 126 and the bill acceptor 138 may have respective internal UV lights which disinfect player cards or bills as they are inserted. The buttons in the button panel 130 can also have internal UV lights which shine through a transparent or translucent surface of the button to disinfect its surface. In some implementations of the present disclosure, an EGM can be equipped with a UV light wand that a player manually passes over the EGM to disinfect it. In other implementations of the present disclosure, a UVGI device may be integrated as close to a surface as possible in order to decrease the duration needed to effectively kill any microorganisms such as viruses or bacteria. For example, the light bar 160 may be located directly over the button panel 130, and another light bar may be integrated into a trim piece around the display 116 and be configured to shine on and disinfect the lower half of the display 116 which often contains touchscreen controls for adjusting wagering denominations and game settings.

A UVGI device can be attached to an EGM using a mechanical assembly that allows the device to be hidden until activated or move around an EGM to cover additional surface area in accordance with the present disclosure. For example, a UVGI device can be hidden in a trim piece of the button panel 130 and, once activated, extended upward, via gears or a spring, to allow a UV light to shine over the button panel 130. In some implementations of the present disclosure, a single UVGI device may be used to disinfect a bank of two or more EGMs. The UVGI device can be attached to a track which extends the length of the EGM bank or a movable arm which allows the device to travel over each EGM in the bank. The UVGI device continually or periodically moves across each EGM in the bank and can be controlled manually or through software to move to a specified EGM in the bank as needed. For example, the UVGI device may be connected to system software which can detect when an EGM needs to be disinfected and instruct the UVGI device to locate itself over the EGM and activate a UV light.

UVGI devices may be activated manually by casino personnel or automatically by software of an EGM or casino management system (“CMS”) which monitors for conditions indicating that disinfection should be performed in accordance with the present disclosure. A UVGI device can be equipped with a local and/or remotely controlled switch or solenoid for activating and deactivating the UVGI device. Casino personnel, the EGM, or a CMS can activate the UVGI device once conditions for triggering disinfection of an EGM are detected. These conditions can include, for example, an elapsed period of time since the EGM was last disinfected, game events such as a player cashing out of an EGM, a threshold number of activations (such as spins), or players at an EGM, etc. Additionally, an EGM can be equipped with sensors or devices which gather data for determining whether disinfection should be performed. For example, a non-contact thermal sensor or infrared camera can be used to determine if a player at an EGM has a high temperature indicating that the player may have an infection. Once the potentially infected player cashes out, the EGM or CMS software automatically activates a UVGI device to disinfect the machine. A UVGI device may be activated opportunistically when it is detected that no humans are within proximity of an EGM. Motion sensors, such as ultrasonic and infrared devices, can be integrated into an EGM and used to determine the proximity of any humans to a machine. Other devices, such as Bluetooth or Wi-Fi radios, may be used to detect whether any phones or other personal electronics, and therefore people, are nearby.

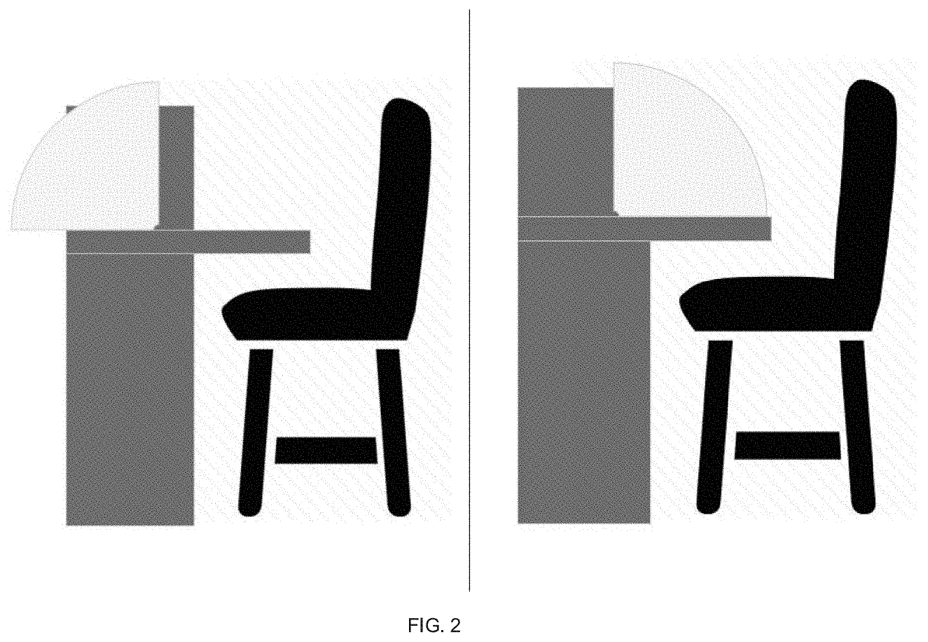

In addition to detecting the proximity of patrons, other protections may be put into place to prevent exposure of patrons to harmful UV light in accordance with the present disclosure. The service light of an EGM can be activated while disinfection is pending, about to begin, or being performed, and the EGM can emit a noise, such as a siren or voice recording, to warn patrons to stay away from an EGM. The service light may be used to indicate the progress of disinfection. For example, a red light may indicate that an EGM is undergoing disinfection, a yellow light may indicate that the disinfection process is almost complete, and a green light may indicate that the EGM is ready for use. The EGM display can show a warning message and a countdown timer for the disinfection process to indicate when it will be safe to approach the EGM. Additionally, a shroud or barrier can be placed around an EGM or a portion of the EGM being treated with harmful UV light. A barrier, such as a temporary wall, can be manually put in place by casino personnel, or EGM or CMS software can activate a barrier such as motorized curtains which surround one or more EGMs when closed. In some implementations of the present disclosure, a shroud may be integrated into an EGM. For example, an EGM can be equipped with a motorized plastic covering which folds over a button panel or other portion of an EGM to block any UV light from a player. FIG. 2 depicts one example of a covering that may be motorized or manually operated by a player. The EGM can be equipped with a sensor to detect whether the covering is in place prior to activating any UV lights.

EGMs can be equipped with a sensor to detect the current or cumulative amount of UV light to which the EGM and the area surrounding the EGM has been exposed in accordance with the present disclosure. If the sensor detects that the current amount of UV light in an area is too intense and may be harmful to humans, the EGM can immediately disable any active UV lights, emit a warning sound, or display a warning message to a player. The sensor can also detect if the cumulative amount of UV light at the EGM over a period of time exceeds a recommended safety threshold. In such an event, the EGM can notify a player and then disable itself until a sufficient amount of time has passed to render the EGM and surrounding area safe. If an EGM is equipped with an always on UV light, the sensor can begin recording a cumulative amount of emitted UV light from the time a player begins playing at an EGM. The EGM can display a countdown timer or other message indicating how much time a player has until they have been exposed to unsafe levels of UV light.

To further reduce the chances of over exposure, the intensity of a UV light can be modulated based on current conditions around an EGM in accordance with the present disclosure. The intensity of a UV light may be modified through pulse width modulation or by controlling the amount of power provided to the light. If a player is currently sitting at an EGM, the UV light can be set to a low intensity setting. If an EGM detects that no patrons are within a specified proximity of the EGM, the UV light can be set to a high intensity setting. The intensity setting can vary automatically based on the proximity of any patrons.

To power the UVGI devices, the UVGI devices may have a separate power source or may draw power from an EGM power supply in accordance with the present disclosure. For example, a UVGI device can be plugged into a USB port of an EGM for power or can be wired into the power supply of the EGM. UVGI devices may also have their own power supply that is separate from the EGM's power supply. Multiple UVGI devices, either on one EGM or across EGMs in a bank, can share a single power supply and alternate powering on to ensure sufficient power is available. In instances where a UVGI device covers a bank of EGMs, the device may selectively draw power from EGMs in the bank which are not currently in use or which have been powered down or put into a low power mode. The selective power draw may be needed to prevent overloading of an EGM power supply for an EGM that is in use. Intensity controls can also be used to reduce the power draw of a UVGI device. For example, if an EGM or multiple EGMs in a bank are currently active, the UV light can be set to a low intensity setting to reduce the amount of power used and prevent overloading a power supply.

In addition to the above embodiments, UVGI devices can take the form of portable battery powered or plug-in devices which can be moved to EGMs as needed for disinfecting in accordance with the present disclosure. For example, a plastic cover can be custom fit to an EGM's button panel shape and equipped with interior UV lights and a battery. The custom fit of the plastic cover prevents UV light from bleeding out and harming humans which the device is placed on an EGM for disinfection. Additionally, any of the described UVGI devices can be designed to be tamper proof or account for activities in the gaming environment. For example, a UVGI device such as the light bar 160 may be designed to be easily broken away from and removed from a machine when force is applied. The force for breaking the light bar away should be sufficiently low so that a patron is unable to pull a machine over or tip it by pulling on the light bar. Additionally, UVGI devices integrated into an EGM may be waterproof to prevent damage from spilled drinks.

UVGI devices can be used in associated with other products besides EGMs in accordance with the present disclosure. UVGI devices may be attached to lottery vending machines, ATMs, gaming tables for table games, chairs used at EGMs and tables, cashier counters, chip trays for table game chips, etc. For example, UV lights may be integrated into the railing of a gaming table. Furthermore, some traditional products can be redesigned to allow for simpler disinfection. EGMs may be modified to limit the number of discrete components such as buttons or trim pieces so that the EGMs includes smoother more uniform surfaces which can be easily wiped down or exposed to UV light. Gaming tables and chip trays can be made of glass or other transparent material to allow UV light to shine through from underneath the table to disinfect the surface and any gaming chips on the table.

Other disinfecting devices, substances, or techniques may be used in place of or as a complement to UV light in accordance with the present disclosure. For example, EGMs can be heated or steam cleaned at a specified temperature for a sufficient period of time to kill any germs. Additionally, parts or all of the EGMs may be coated with naturally antimicrobial materials. For example, EGM screens may be infused with ionic silver or coated with titanium dioxide. EGMs can also include automatic sanitizer spritzers.

### Reservations and Optimizing Available Games

In order to comply with occupancy restrictions or to enforce social distancing, casinos may be required to take measures which limit the amount of available EGMs or betting spots at table games in accordance with the present disclosure. For example, the casino may disable alternating EGMs to create space between players, limit the number of players at table games, or divide the casino floor into zones and limit the number of people in each zone. The casinos can improve the player experience in such circumstances by, for example, allowing patrons to reserve particular EGMs or table games and optimizing available EGM game titles to satisfy player demand.

For reservations, casinos can allow players to reserve games via a website or smartphone application or allow a player to call or text a phone number of the casino to indicate which game the player wishes to play in accordance with the present disclosure. Additionally, the casino can predict which game or games a player is likely to play and automatically reserve the game for that player. For example, the casino can determine which games a player is likely to play based on their play history and reserve that game once the player's arrival is detected. The casino may detect arrival of the player based on the player carding into an EGM or kiosk, the player's phone connecting to casino Wi-Fi, or the player checking in through an application. The player can be notified through an application or text message of the location of their reserved machined. A machine which is reserved for a player may be locked or disabled until the player arrives and inserts his or her player's card into the EGM.

Using reservations data or through real time tracking of players, casinos can determine which games are in demand and dynamically adjust available games in accordance with the present disclosure. For example, if 10 players have reserved Game A and only 7 EGMs are currently operating Game A, the casino may convert 3 EGMs to instead run Game A. Similarly, if real-time data shows that 90% of EGMs with Game B are currently in use and only 30% of EGMs with Game C are in use, the casino may convert a percentage of EGMs with Game C to instead run Game B. The process of optimizing available games may be automated by a CMS or other software.

Additionally, casinos can dynamically adjust available games or intelligently select EGMs for reservations to ensure a uniform physical distribution of patrons in accordance with the present disclosure. As reservations are received, the casino selects EGMs to reserve based on their locations on the casino floor and the locations of other active or already reserved EGMs. If a patron wishes to play a game that is only available in an already crowded portion of the casino, the CMS can convert an EGM in a non-crowded area of the casino floor to the desired game and reserve that EGM for the player.

Due to occupancy restrictions, some patrons may be forced to queue up for entry into a casino. The queue for entry may be a physical or virtual line. For example, patrons may check in to the line from an electronic device which indicates to the patron a place in line or an estimated time to entry. Patrons may be offered promotions or other incentives based on how long they have been in the queue in accordance with the present disclosure. Additionally, patrons which are waiting may be allowed to play casino games from a mobile device for real or virtual currency in accordance with the present disclosure. Any progress made in a game on the mobile may be synchronized to an EGM once the patron is allowed entry into the casino. In instances where only casino games using virtual currency are allowed on the mobile device, some game data may still be synchronized, such as unlocked features or collected symbols or wilds, even though the credit balance is not synched. Alternatively, if the wait time is prohibitively long or a patron has waited a threshold amount of time, the patron may be notified on their mobile device that he or she may continue playing the game even if the patrons exits the queue for entry.

### Sanitizing Table Chips

Table game chips could be sanitized after use in accordance with the present disclosure. When a player wins, the dealer could distribute new clean chips to the player and the “used” chips could be placed into a slot in the table that catches and cleans chips. The chips might have a sensor to detect that they should be exchanged. This could happen after a certain about of use, such as after 10 minutes. For example, the chip might beep, flash, vibrate or notify the dealer after 10 minutes to exchange the chip for a clean one. The chips could be made of an antimicrobial material or smooth such that they are much easier to clean. The chips could have a clean indicator to enforce confidence in the player. After cleaning the chip could light green and then when they become dirty the chip could light red.

The table might be equipped with a smart drop box where dirty chips are added to the drop box after a hand and clean chips can be obtained from the drop box after the chips have been sanitized in accordance with the present disclosure. The smart drop box could be equipped with a UV or UVC light to clean the chips, spray them with a disinfectant, etc. The smart drop box may be connected to a table-level computer associated with the table management system (ex: IGT's Table Manager) and the table-level drop box can report the chips that are inserted and removed by the dealer since at table close, the drop box may contain chips and that amount needs to be tracked and reported to ensure the dealer is not stealing chips.

Chip sorters can also be equipped with UV light in accordance with the present disclosure. Chip sorters can be enclosed in a closed box which allows the chips to be illuminated with UV so that chips are cleaned while not impacting dealers or other table staff. Similar techniques may be used to sanitize dice and playing cards after use. For example, UV lights can be integrated into automatic card shufflers, and UV sanitization boxes can be placed at tables for sanitizing dice between uses. Card shufflers may also be integrated with UV lights to ensure that each card is exposed to a UV light for the required period of time. This can happen during the shuffling process, or in a separate device that does not shuffle cards but pulls cards from one or more input card decks and exposes the surfaces of each individual card to UV light.

### Remote Control of EGM Using Mobile Device

To reduce physical contact with EGMs, players can remotely control the EGM from their mobile or tablet device as opposed to interacting with a button panel on an EGM in accordance with the present disclosure. A player uses an application on a mobile device to establish a connection with an EGM over which commands such as spin, wager amount, max bet, cash out, etc., can be sent. Additionally, a player can digitally establish a credit balance on an EGM by transferring funds from a player account through the application.

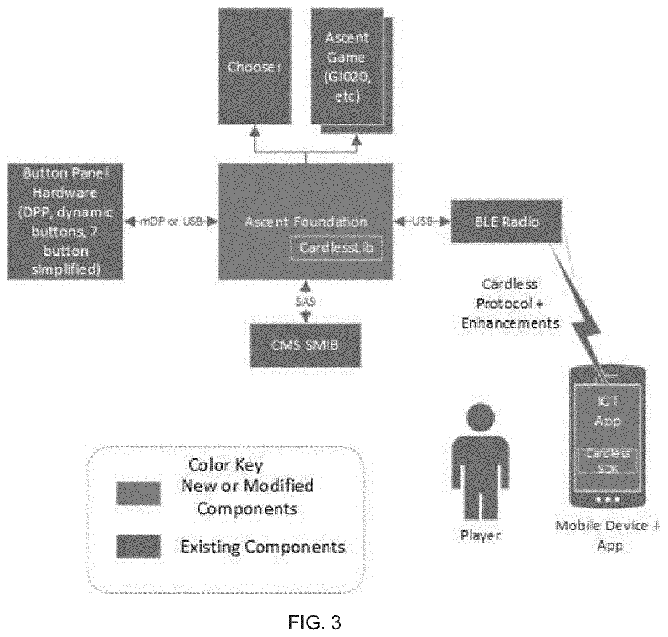

FIG. 3 depicts an example system architecture for enabling a mobile device to control an EGM in accordance with the present disclosure. An application installed on the mobile device is configured to establish a communication channel with a variety of EGMs and transmit commands for controlling a connected EGM. To establish a connection between the application and an EGM, a player may scan a QR code displayed on the EGM, manually enter a unique identifier for the EGM into the application, obtain connection data from the EGM through NFC, or establish a Bluetooth or Wi-Fi connection with the EGM. In FIG. 3, once a connection is established, the mobile device transmits commands to the EGM over a Bluetooth connection which are relayed through an operating system of the EGM to the game software. In some implementations, the EGM can host an HTTP web server or similar protocol which is capable of receiving remote control commands. The application obtains a URL and port information for the EGM web server and transmits commands via a network, such as a cellular or Wi-Fi network, to the EGM. These commands may pass through and be routed by intermediary servers.

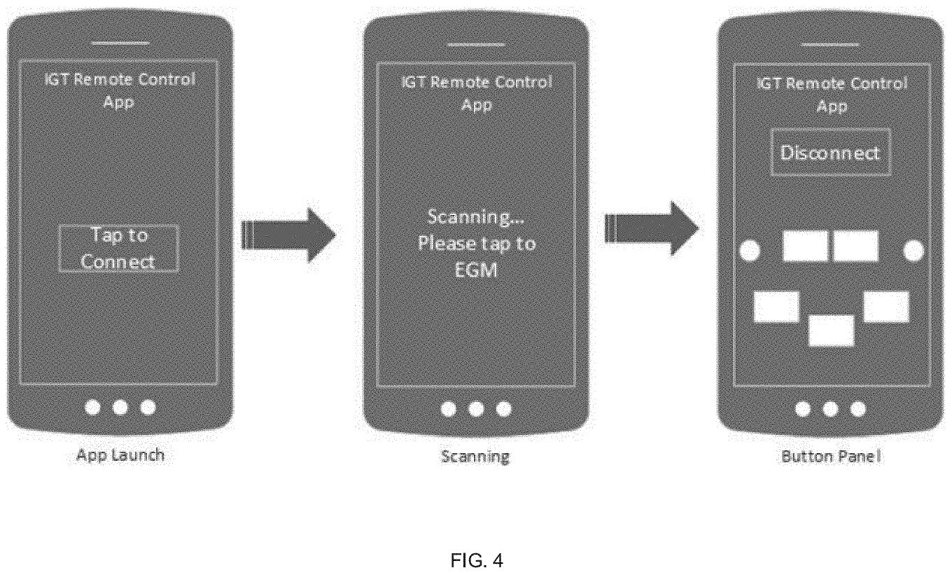

FIG. 4 depicts an example connection process and illustrates on screen instructions presented to a use for establishing a connection between the mobile device and the EGM in accordance with the present disclosure. The final user interface provides a virtual button panel with which a user interacts to remotely control a connected EGM. The application can obtain information from the EGM during the connection process and then customize the displayed button panel to correspond to a particular game, EGM manufacturer, button panel layout, etc. For example, the application may obtain a model number for a connected EGM and then query a database to obtain a button panel layout for that model which is then displayed in the application. The application may also obtain data specific to a game title such as available wagers, themes, graphics, denominations, etc. and then configure the virtual button panel accordingly. To improve the usability of the virtual button interface, not all physical buttons on an EGM may be digitally reproduced. Additionally, the application may allow a user to customize the button panel layout to only include their most frequently used wagering options or add additional buttons not typically available on EGM, such as programmed buttons for customized betting sequences, auto-spin options, high frequency spin button activation option for tournaments, etc.

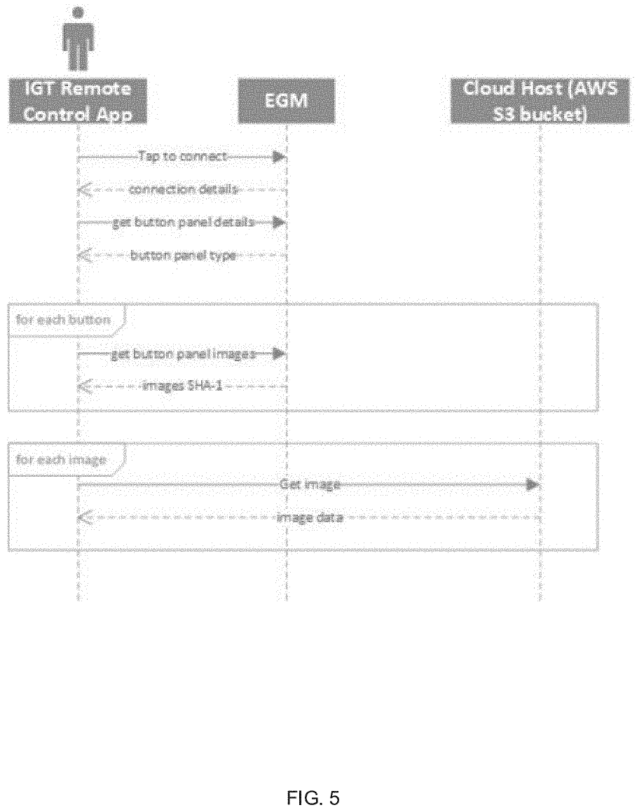

FIG. 5 depicts an example process for supporting EGMs and games with dynamic button variants in accordance with the present disclosure. An EGM with dynamic buttons (e.g. digital player panels (“DPP”) or touchscreens with software buttons) contains multimedia such as images or videos that are unique to the EGM model or game which are displayed on the buttons. Reproducing the multimedia on the virtual button panel can be difficult in instances where the mobile device is connected to an EGM over a low bandwidth connection such as Bluetooth. In some instances, even a high bandwidth connection such as Wi-Fi may be insufficient for reproducing the dynamic buttons if the network is unreliable or an EGM lacks sufficient processing power for encoding and transmitting the images or videos. To remedy these issues, the application can use a unique identifier for the game or multimedia to retrieve the images from a server. As shown in FIG. 5, after obtaining button panel information from an EGM, the application retrieves from the EGM SHA-1 fingerprints or hashes which serve as unique identifiers for the dynamic button multimedia. The application then uses the SHA-1 fingerprints to query a cloud hosted server which contains the multimedia. Using fingerprints or hashes for the multimedia greatly reduces the amount of data which is transferred over the potentially low bandwidth connection between the mobile device and the EGM.

In some implementations, button panels configurations can be identified using photo recognition in accordance with the present disclosure. A player uses their mobile device to take a picture of the button panel at an EGM. The picture can be sent to a server for identification and the server returns configuration information or multimedia for the identified button panel in a similar manner as described above with fingerprints. Additionally, the mobile device or server can use the picture or the mobile device camera to map out and create a virtual button panel. This is especially useful for DPPs or EGMs without physical buttons. The player can take a picture or use an application on a phone or mobile device to scan the DPP and use visual recognition algorithms to identify buttons. These buttons are then mapped to locations and visually represented on the phone to depict the buttons. When a user touches a button on the phone, a touch location from the phone is translated to specific coordinates of the DPP. The coordinates are sent wirelessly to the SMIB which sends a message to the game software that a touch at coordinates x, y occurred.

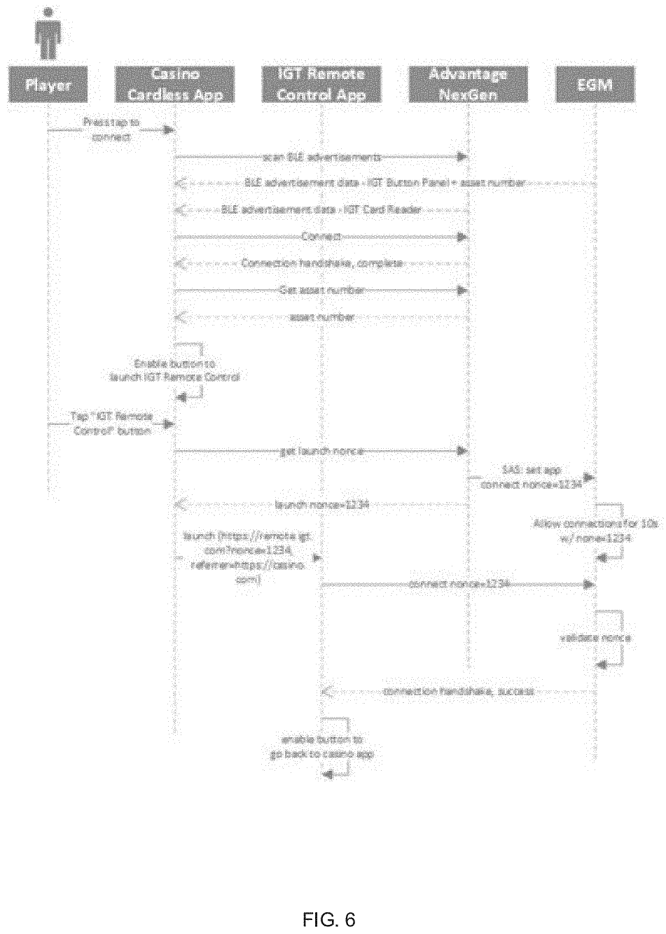

FIG. 6 depicts an example process for validating a connection with a mobile device in accordance with the present disclosure. As part of an initial handshake process, the slot machine interface board (SMIB) on the EGM generates a nonce value or one-time password which is transmitted to the mobile device over a short distance, local communication channel such as Bluetooth or NFC. The application on the mobile device transmits the nonce value and an identifier for the EGM to a backend server which then transmits the nonce value back to the identified EGM over a network connection. If the EGM determines that the nonce values match, the EGM continues with establishing the connection with the mobile device and allowing remote control of the EGM. In place of or in addition to exchanging a nonce value, the EGM may only allow connections with a mobile device that has a high signal strength, which prevents players not sitting at the targeted EGM from hijacking a machine remotely. The requirement for a high signal strength connection may be relaxed or eliminated if a successful nonce value validation has occurred. Additionally, in some implementations, commands transmitted between the mobile device and the EGM may be encrypted using a one-time key or a public-private key pair which are exchanged during the connection process. Encrypting the commands provides additional security and prevents harmful actors from spoofing commands for a player's EGM.

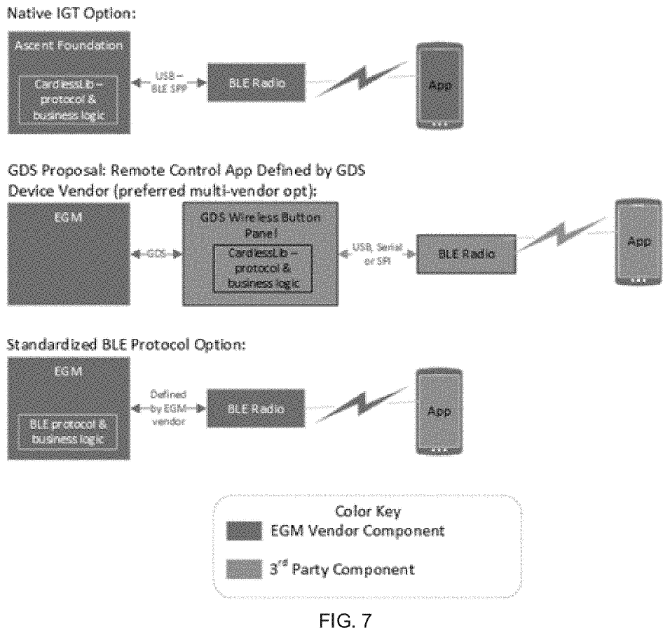

FIG. 7 depicts systems for supporting remote control of EGMs from multiple manufacturers in accordance with the present disclosure. In one system, a standardized Bluetooth protocol is implemented that allows a remote control application to work seamlessly across different manufacturers. In another system, EGMs from manufactures which do not natively support remote control over a wireless connection are equipped with an intervening device that relays commands to game software on the EGM. For example, the intervening device may be a device using the Gaming Standards Association's Gaming Device Standards (GDS) protocol, such as wireless button panel which receives commands over a wireless connection and then converts the commands into simulated hardware signals. The converted commands are transmitted to the game software of the EGM and treated by the software as if they originated from a typical physical button panel.

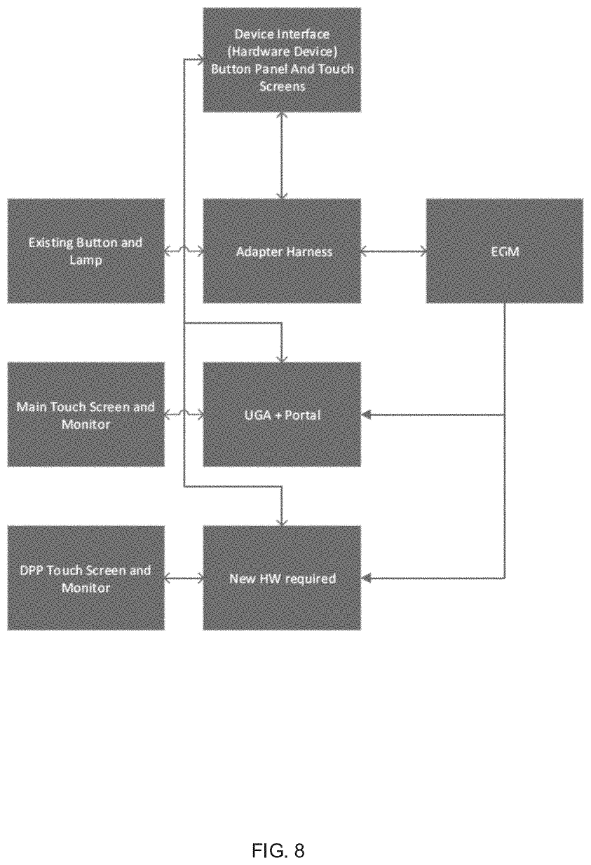

FIG. 8 depicts an additional high-level architecture view for supporting remote control of EGMs from multiple manufacturers in accordance with the present disclosure. This architecture introduces additional hardware to enable remote control. The additional hardware can be wired into existing button and lamp wiring, powered over USB, or given their own power supplies.

Many architecture variations for remote control of an EGM are possible in accordance with the present disclosure. An EGM can be equipped with multiple SMIBs to handle different operating systems from different machine manufacturers. To implement a floor wide, manufacture agnostic solution for a remote control mobile application, a second SMIB or board can be added to games that could interface to any machine processor. The commands would be transmitted to the EGM similar to the architecture flow shown in FIG. 4; however, the original SMIB would communicate with the added SMIB which would be customized to talk to the processor of that particular operating system/cabinet.

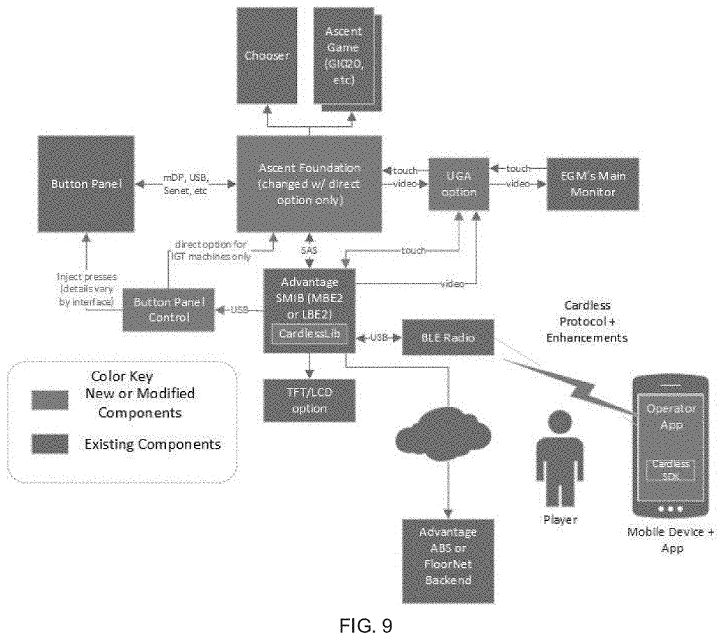

FIGS. 9, 10, 11, 12, and 13 depict additional architectures for enabling remote control of an EGM from a mobile device in accordance with the present disclosure. These different architectures address the variations of button panels, DPPs, and other EGM controls found in the marketplace. FIG. 9 depicts a high-level architecture where players can remotely control an EGM button panel via a mobile application (referred to as Cardless in the figure). The architecture diagram shows that button panel control hardware is added to work with the traditional SMIB and interface with the EGM's button panel. Additionally, universal game adapter (UGA) hardware is added to interface with the EGM's main touchscreen to allow injection of touches without requiring the player to physically touch the touchscreen monitor. Although depicted as discrete components, in some implementations, the SMIB firmware can be upgraded to perform the functionality of the UGA or button panel control hardware, thus eliminating the need for additional devices.

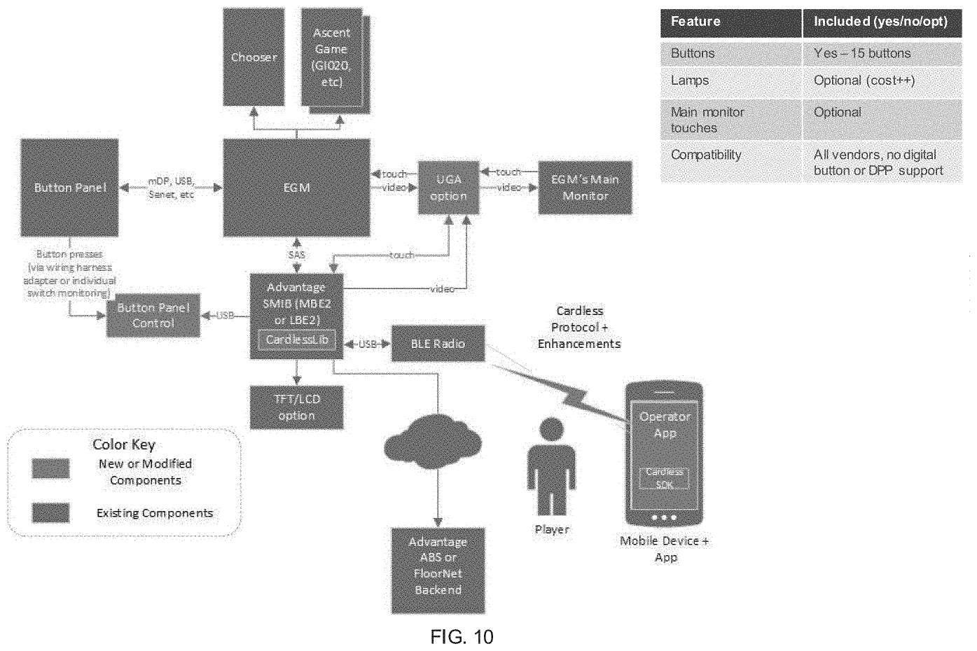

FIG. 10 depicts an architecture for enabling remote control of an EGM with a traditional wired switch button panel in accordance with the present disclosure. A new UGA-like device (“Button Panel Control”) is used to integrate the SMIB with button panel. The Button Panel Control interfaces with the SMIB over USB. This approach does not require any modifications to the EGM.

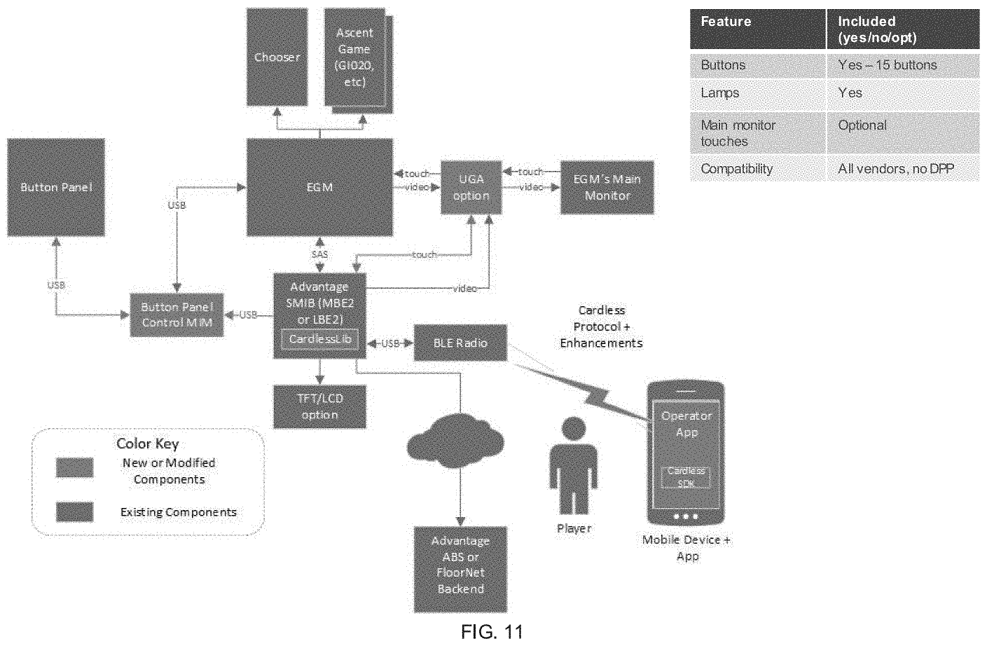

FIG. 11 depicts an architecture for enabling remote control of an EGM with button panels which interface via USB with EGM in accordance with the present disclosure. This architecture employs a man-in-the-middle type approach by adding a device between the button panel and the EGM to allow for injection of button commands received from the mobile device. The Button Panel Control device utilizes the same protocol as the button panel to emulate commands as if they originated from the button panel.

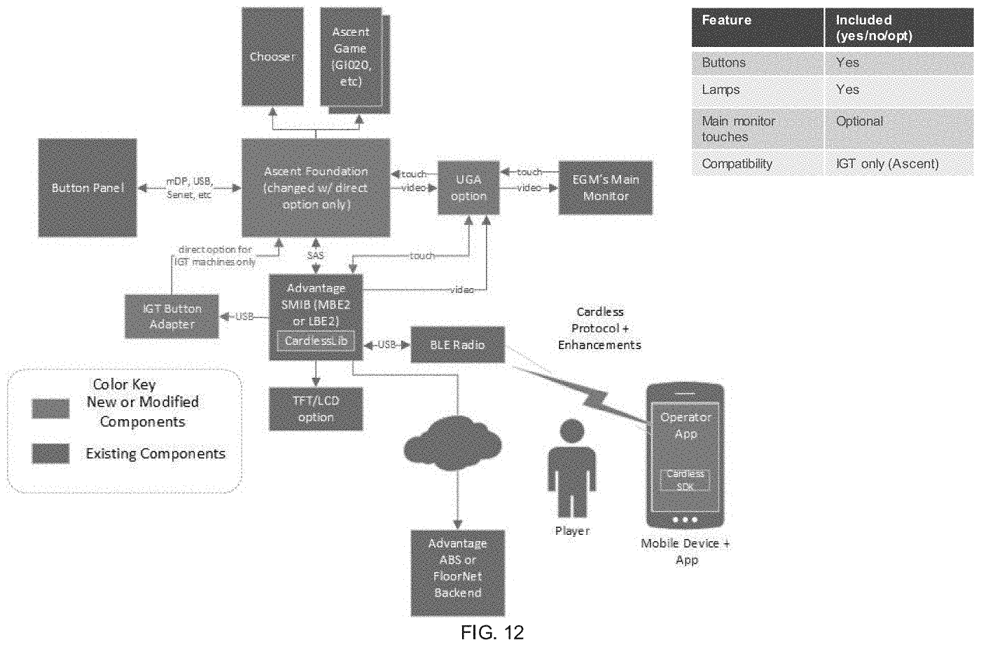

FIG. 12 depicts an architecture for enabling remote control of an EGM through a direct connection with the EGM from the SMIB in accordance with the present disclosure. In this system, the operating system of the EGM (referred to as Ascent Foundation in the figure) is modified to allow the injection of touches or button commands directly from the SMIB, thus eliminating the need for intermediary devices. Additional hardware can be added to support additional USB connections or, as shown in the figure, a button adapter can be added to enable a USB connection to the EGM through various button port types.

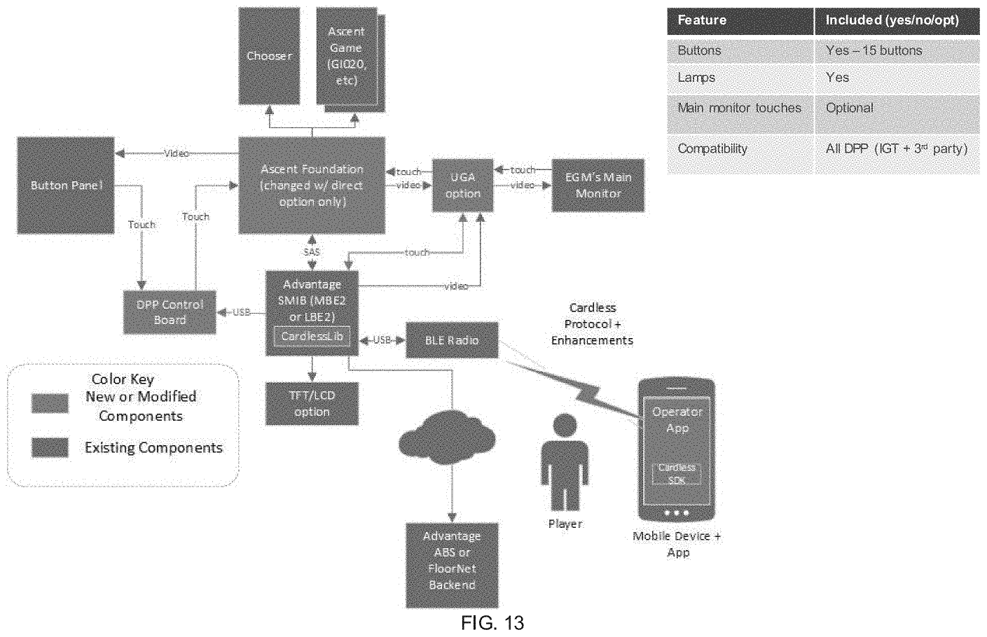

FIG. 13 depicts an architecture for enabling remote control of an EGM with a DPP button panel from third party manufacturers in accordance with the present disclosure. This system also uses a man-in-the-middle type approach with a DPP control board device that allows for injecting touches from the mobile device. This approach can also allow the DPP on the EGM to be updated with visual cues for the software buttons as if the player had touched the buttons instead of sending commands via their mobile device. For example, a button on the DPP may typically change colors when pressed by a player. Using this technique, a player pressing a virtual button on his or her mobile device can see that input reflected on the DPP when the EGM changes the color of the corresponding button. This is possible since the DPP control board emulates touches as if they originated with the DPP button panel. In this architecture and in other man-in-the-middle approaches described above, the control board relays any touch commands or button commands which originate from the traditional button panel without modification.

Although the above examples describe wireless control of an EGM via a mobile or tablet device, other devices may be used to control an EGM in accordance with the present disclosure. For example, a game controller for a gaming console or PC may be wireless or physically connected to an EGM and the buttons of the controller mapped through software to replicate button panel functions. An EGM can be configured to accept a detachable button panel that a player brings with them from home or checks out from the casino. The detachable button panel can be returned to the casino after use for sanitization. The detachable button panel may connect through a USB port which is accessible by the game software or connect wirelessly or physically to a SMIB which routes commands to the game software.

Additionally, EGMs may be controlled through a combination of eye tracking and gestures in accordance with the present disclosure. For example, a gesture like ‘pushing your hand down’ like you are pressing a button can be detected by a gesture motion sensor and then associated with the direction of the player's gaze. Currently in many eye-tracking applications, the player has to stare at a location for a couple seconds in order to activate a button. This is tedious and time consuming and has a high failure rate. With gestures, you have to be pointing at a specific point and then do the gesture. As you move your hand to the edge of the sensor, the position accuracy of detecting the gesture lessens; and the player needs to track where they are pointing and then do a gesture without losing the sensors detection. By combining eye-tracking and gestures, the player can do a simple, intuitive motion anywhere in the sensors range and associate it to where the player is looking. This is a quick detection and easier to use.

### Adjacent EGM Disablement Using Wireless Proximity Sensors

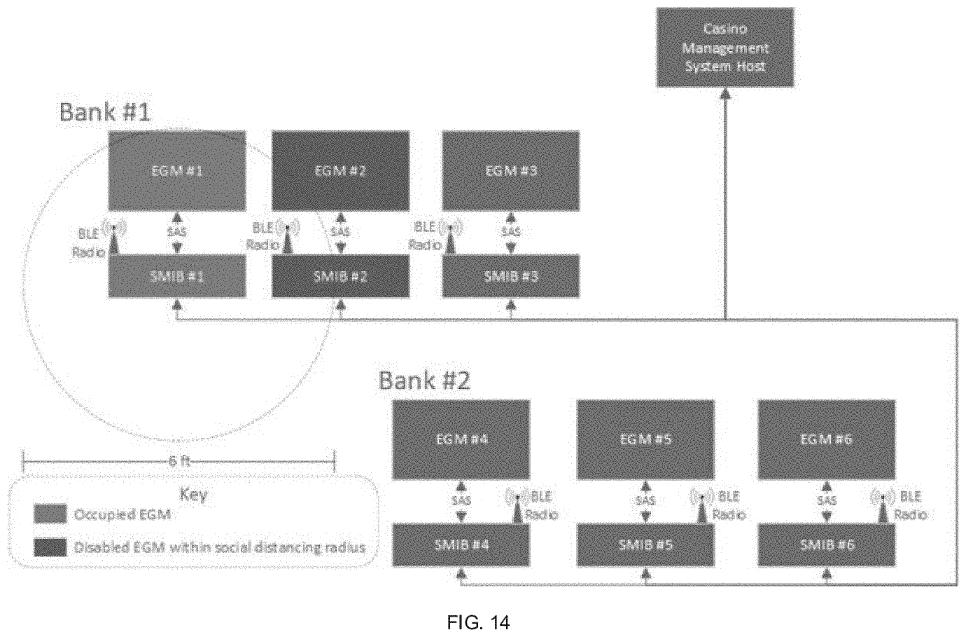

As noted above, one approach to implement social distancing in a casino is to disable every other machine, but that presents problems to the players and potentially to a gaming jurisdiction. First, social distancing artificially limits the game selection across the casino floor, thereby making it more likely that some players may leave the casino after not being able to find their desired game. Secondly, it presents tax revenue challenges to certain jurisdictions, such as the state of Nevada, which receive a tax payment per operational EGM per month. Software of the CMS, EGM or SMIB can be enhanced in accordance with the present disclosure to: (1) Detect adjacent EGMs that are within social distancing range when a player starts a play session at an EGM; (2) Communicate to the CMS adjacent EGMs to the EGM where a player is starting a player session; (3) Have the CMS disable those adjacent EGMs; and (4) Have the CMS re-enable those adjacent EGMs once the player's session ends. FIG. 14 depicts a high-level architecture view of the system for detecting adjacent EGMs to be disabled in accordance with the present disclosure.

Other approaches are possible where a CMS can determine directly adjacent machines by interpolating adjacent EGMs from a subset of each EGM's machine location field that is tracked by the CMS in accordance with the present disclosure. This approach, however, requires casino operators to maintain the machine location field across machine movements and placement changes, which may result in the field containing stale data. Additionally, machine location fields may imply that one EGM is adjacent to another EGM, when it may be separated by some distance. As a result, relying upon this field to determine adjacent EGMs may have unintended side effects and result in operator frustration. Alternatively, actual proximity data extracted across the slot floor in real time (or near real-time) based upon the historical monitoring by the EGM at which a player starts a play session can be leveraged to determine which adjacent EGMs to disable to support social distancing requirements. Another approach is to save in some data storage the set of adjacent EGMs to disable. This could be entered and saved in the back-end of the CMS, or entered by a technician in the SMIB and reported to the back-end of the CMS when the SMIB comes online, or at runtime when a player session starts. This information can then be leveraged by the CMS to disable adjacent EGMs when a player session starts, and re-enable them when a player session ends. The downside of this approach, much like the floor location-based approach, is that the information needs to be properly managed by the casino operator in order for the social distancing to function correctly and as desired (in other words, that the system is disabling and re-enabling the intended EGMs).”

To determine EGM proximity data, an EGM or SMIB may use wireless radios, such as Bluetooth, wi-fi, or NFC, to determine, via signal strength information, which EGMs are approximately within social distancing range of a given EGM in accordance with the present disclosure. Additionally, techniques can be used to combat possible security threats or attacks that malicious players may attempt to perform on the solution in order to trick the system to disable EGMs which are not actually adjacent to an active EGM.

### Detecting of Adjacent EGMs

In this embodiment, the EGM or SMIB is equipped with a wireless receiver and transmitter of some sort in accordance with the present disclosure. The EGM or SMIB will use this transmitter or receiver to perform the following: (1) Listen for adjacent EGMs using the receiving functionality of the radio; and (2) Broadcast its location to other adjacent EGMs using the transmitting functionality of the radio. The EGM or SMIB will adjust from one mode to another (broadcasting or receiving) based upon its current state or based upon a time interval, or the wireless radio may support operating in both modes simultaneously. The radio may also be capable of performing both operations concurrently (broadcasting and receiving). In another embodiment, the EGM or SMIB may be equipped with one dedicated wireless receiver and another dedicated wireless transmitter but separating out the functionality across multiple pieces of hardware will result in added costs.

In one embodiment, the EGM or SMIB may detect adjacent EGMs when it detects a player occupying event, or a player session start event in accordance with the present disclosure. The section “Detecting a Player Occupying Event” below provides more information about how an EGM or SMIB can detect a player occupying event. In another embodiment, the EGM or SMIB may periodically listen for adjacent EGMs or SMIBs and maintain a list of adjacent EGMs so that the list of adjacent EGMs or SMIBs does not have to be determined at runtime when a player occupying event is detected as the real-time detection of adjacent EGMs may add unacceptable latency and impact the overall player experience. Furthermore, it is possible that wireless signal strength may vary over time based upon changes in the ambient environment or attempts by players to disrupt the overall application behavior with tools such as signal repeaters, etc. In order to combat such risks or threats, the process of background scanning for adjacent EGMs or SMIBs may be built in such a way to require a consistent signal strength threshold for a period of time before an adjacent EGM can be added to the list (such as 10 minutes). The threshold could be hardcoded or configured through the EGM, SMIB, or configured in the CMS and the configuration transferred to the EGM or SMIB at startup or runtime. In another embodiment, the threshold may not be fixed and rather require the EGM or SMIB to perform an attenuated calculation where each EGM or SMIB may publish the expected signal strength at 1 meter distance from the cabinet and the receiving EGM or SMIB may adjust the received signal strength to normalize the distance to the adjacent EGM based upon differences in signal strength as a result of varying slot machine cabinet designs as the published expected signal strength allows a recipient of a broadcast to compensate for those factors when determining proximity. If a player engages with the EGM or SMIB using a wireless communications protocol, such as IGT's Bluetooth Low Energy (BLE) based Cardless Connect protocol, then the EGM or SMIB may temporarily disable its background process to monitor or track adjacent EGMs until the Cardless Connect connection is completed and instead the EGM or SMIB may rely upon the list of adjacent EGMs gathered previously, before the current Cardless Connect connection was established by a player.

When a player session occupying event is detected, the EGM or SMIB must inform the CMS that a player occupying event has been detected and also inform the CMS about adjacent EGMs that have been detected so that the CMS can then disable those adjacent EGMs. In one embodiment, the EGM or SMIB may send an event or message to the system with a list of the current adjacent EGMs. In another embodiment, the EGM or SMIB may send an event or message to the CMS for each adjacent EGM in the list of adjacent EGMs or SMIBs. In another embodiment, the EGM or SMIB may continuously share its list of adjacent EGMs or SMIBs with the CMS so that the list doesn't need to be included with the player session occupying event or with a series of player session occupying events when they occur.

Upon receipt of this message or event or set of messages or events, the CMS will then perform basic validation checks on the EGMs or SMIBs listed as adjacent. For example, the CMS may ensure that the EGMs or SMIBs listed are valid, registered, and/or currently online with the CMS. If the validation checks pass successfully, then the CMS will send a message to each adjacent EGM or SMIB, informing that EGM or SMIB to disable itself. In another embodiment, the CMS may not disable the adjacent EGMs, but may instead change a user interface at the adjacent EGMs to describe them as not safe to use.

At the end of a player's session, the EGM or SMIB must coordinate with the CMS to re-enable adjacent EGMs or reset any “unsafe to use” messaging that may appear at adjacent EGMs. A player session end can be detected by the detection by the EGM or SMIB of a player departing event. Upon detection of a player departing event, the EGM or SMIB will inform the CMS by sending it an event or message that it has detected a player departing event. The player departing event may describe the list of adjacent EGMs that were disabled when the EGM or SMIB originally detected a player occupying event. In an alternative embodiment, a player departing event may only describe one adjacent EGM and a unique player departing event or message could be generated for every adjacent EGM that was disabled when the EGM or SMIB detected a player occupying event. In an alternative embodiment, the player departing event message sent by the EGM or SMIB to the system may describe zero adjacent EGMs with the expectation that the CMS maintains the list or set of adjacent EGMs that need to be re-enabled after a player departing event is processed from that EGM or SMIB.

Some scenarios may exist where the EGM or SMIB may go offline and a player departing event may never get to the CMS so that adjacent EGMs can be disabled. This can occur if the initiating EGM had a power failure or malfunction that rendered it inoperable and unable to report a player departing event. In order to ensure that other adjacent EGMs eventually become re-enabled, the CMS may require a heartbeat message (or series of heartbeat messages or events) to be published by the initiating EGM or SMIB to maintain the disabled state of the EGMs adjacent to the initiating EGM. If a required heartbeat(s) are missed, then the CMS may re-enable one or more disabled or locked adjacent EGMs, or may adjust the player messaging on adjacent EGMs to no longer describe them as unsafe to occupy.

### Detecting a Player Occupying Event

A player occupying event can occur upon a detection of any one or more suitable detectable actions by a player in accordance with the present disclosure.

In one embodiment, a player occupying event can be detected by a player inserting their player's club card into a card reader attached to the EGM or SMIB in accordance with the present disclosure. The detection of a player occupying event could optionally also require player activity (such as described herein). For example, the player activity requirement could require the player to place a minimum bet to avoid scenarios where players may take advantage of the situation and purposefully disable machines by inserting a card into an EGM, immediately removing it, and moving to the next EGM.

In another embodiment, a player occupying event can be detected by detecting that a player has inserted cash (bills, coins, etc.) or a ticket into the EGM in accordance with the present disclosure. This could be detected directly or indirectly by monitoring the movement of the meters of the EGM. The detection of the player occupying event could optionally require the player to additionally place a minimum bet to avoid scenarios where players may take advantage of the situation and insert a bill or ticket, then cash-out and move to the next EGM, but not place a bet, in an effort to purposefully disable machines throughout the casino floor.

In another embodiment, a player occupying event can be detected through the use of motion sensors coupled to the EGM or SMIB in accordance with the present disclosure. If motion is detected within range of the sensors, or a designated subset of sensor range, then a player occupying event can be generated. This may additionally require the player to place a minimum bet to avoid scenarios where players may take advantage of the situation and briefly sit in front of an EGM to purposefully disable a machine, and then get up and move to the next machine.

In another embodiment, a player occupying event can be detected through use of a camera (traditional or infrared) that detects if a player has occupied the EGM in accordance with the present disclosure. This can be detected based upon monitoring the complete field of view of the camera, or a configured sub-set of the field of view. This could optionally require the player to additionally place a minimum bet to avoid scenarios where players may take advantage of the situation and sit in front of an EGM to purposefully disable the machine, and then get up and move to the next machine.

### Detecting a Player Departing Event

A player departing event can occur upon a detection of any one or more suitable detectable actions by a player in accordance with the present disclosure.

In one embodiment, the EGM or SMIB can detect that a player has left an EGM and that it needs to be sanitized through detection of the player removing their card from the card reader attached to the EGM or SMIB in accordance with the present disclosure. If the player initiated their session using a wireless mechanism (Cardless Connect, etc.), then lack of a signal, or a signal strength below a configured threshold, or a disconnect of the wireless communications channel may also result in the triggering of a player departing event.

In another embodiment, lack of player activity for a period of time (ex: 3 minutes) may result in detection of a player departing event as the player may have left the EGM without removing their player tracking card, or by properly closing the wireless communications channel between their mobile device and EGM/SMIB. Lack of player activity can be detected by the player not interacting with the EGM, by not playing a game, by not pressing the cash-out button, by not triggering any activity that causes the EGM to generate an event or move machine meters, etc.

In another embodiment, the EGM or SMIB can be connected to motion sensors that can detect that a player who was sitting at the EGM has departed. This can be detected by lack of player motion within a given time period.

In another embodiment, the EGM or SMIB can detect that a player has left the EGM by a player pressing the cash-out button to have the EGM print a ticket or to transfer their funds on the credit meter to a cashless system. The solution may trigger a departing player event immediately, or it may wait for up to a given timeout value, as the player at the EGM may not actually leave and may instead insert cash or additional funds into the EGM in order to start a new play session.

In another embodiment, the EGM or SMIB may generate a player departing event after the EGM's credit meter has gone to 0. The solution may trigger a departing player event immediately, or it may wait for up to a given timeout value, as the player at the EGM may insert cash or additional funds into the EGM in order to start a new play session.

In another embodiment, the EGM or SMIB can be connected to a video or infrared camera that can detect if a player is no longer in front of the EGM. This camera can detect this by determining if a player is not located within a certain field of view or area covered by the camera. The solution may trigger a departing player event immediately, or it may wait for up to a given timeout value before generating a departing player event.

### Example Flowcharts

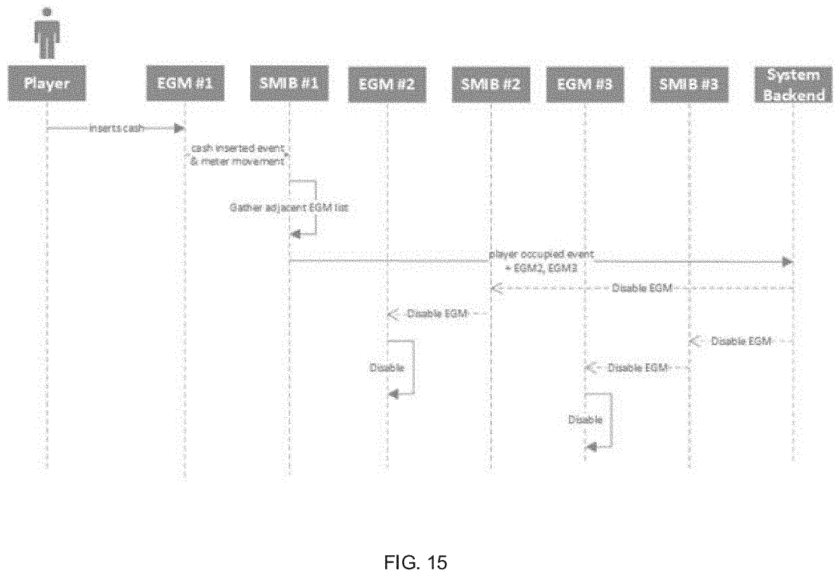

FIG. 15 illustrates one example sequence where the EGM or SMIB describes the set of adjacent EGMs in a single message in accordance with the present disclosure. This sequence diagram assumes that the EGM or SMIB is maintaining the list of adjacent EGMs or SMIBs as a background process, although that is not required.

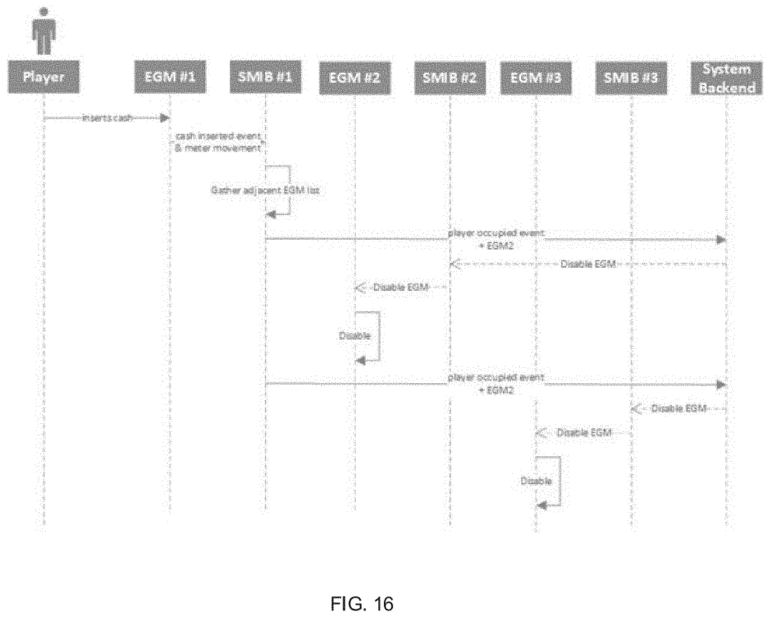

FIG. 16 illustrates one example sequence diagram where the EGM or SMIB describes the set of adjacent EGMs in a message or event per adjacent EGM or SMIB in accordance with the present disclosure. This sequence diagram assumes that the EGM or SMIB is maintaining the list of adjacent EGMs or SMIBs as a background process, although that is not required.

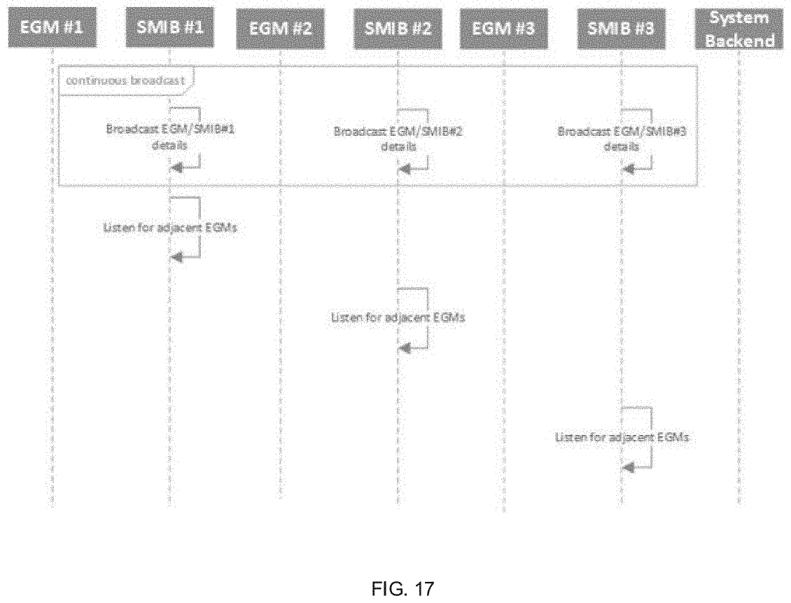

FIG. 17 illustrates the background process of periodically broadcasting the EGM or SMIBs information so that it can be gathered by adjacent EGMs or SMIBs for maintaining their own adjacent EGM or SMIB list, while also listening for adjacent EGMs or SMIBs to maintain its local list of adjacent EGMs or SMIBs in accordance with the present disclosure. The radio can be configured to continuously broadcast EGM/SMIB details in the background, so the SMIB or EGM does not have to specifically trigger the broadcast

### EGM Disinfection Process Automation

Player's concerns that touching an EGM may not be safe due to possible microorganisms such as a virus being on the surface of the EGM can be addressed by enhancing the EGM or Casino Management System to enforce a workflow where an EGM is flagged or disabled after use by a player and then flagged or re-enabled after it has been sanitized by a casino staff member in accordance with the present disclosure. These valuable visual indicators help casinos by enforcing a workflow that helps local policymakers feel comfortable about re-opening casinos and gives valuable indicators to players so that they know which EGMs they can safely use and which EGMs they cannot be safely use.

The following workflow can be enforced by the Casino Management System (CMS) or the EGM: (1) Detecting that a player is at the EGM via detection of a player occupying event; (2) Detecting that a player has left the EGM via detection of a departing player event; (3) Based upon detection of a player departing event, changing the state of the EGM or SMIB to indicate to the player that the EGM needs to be sanitized; (4) Notifying the CMS that the EGM needs to be sanitized; and (5) Upon detection of a sanitization complete event by a casino staff member, changing the state of the EGM to display an indicator that a player can safely play the EGM. The resident CMS may also be notified of the state change. The following sections will discuss the embodiments for each operation described above.

### Detecting a Player Occupying Event

A player occupying event can occur upon a detection of any one or more suitable detectable actions by a player in accordance with the present disclosure.

In one embodiment, a player occupying event can be detected by a player inserting their player's club card into a card reader attached to the EGM or SMIB in accordance with the present disclosure. The detection of a player occupying event could optionally also require player activity. Player activity can be any suitable level of player activity such as but not limited to: (a) the player also inserting cash or tickets into the EGM; (b) the player touching one or more buttons on the EGM's button panel; (c) the player touching one or more areas of the touchscreen input device of the EGM; (d) the player pulling a wager handle on a side of the EGM; (e) the player using a service window displayed by the EGM; (f) the player making a wager to player or playing at least one play of a game of the EGM; and or other suitable game play related events. For example, the detection of a player occupying event could optionally also require player activity in the form of the player placing a minimum bet to avoid scenarios where players may take advantage of the situation and purposefully disable machines by inserting a card into an EGM, immediately removing it, and moving to the next EGM. It should also be appreciated that one or more of the player activities could be the determined to be, by itself or themselves, the player occupying event.

In another embodiment, a player occupying event can be detected by a player pairing the player's mobile device with the EGM or SMIB in accordance with the present disclosure.

In another embodiment, a player occupying event can be detected by detecting that a player has inserted cash (bills, coins, etc.) or a ticket into the EGM in accordance with the present disclosure. This could be detected directly or indirectly by monitoring the movement of the meters of the EGM. The detection of the player activity can be in the form of the player occupying event could optionally require the player to additionally place a minimum bet to avoid scenarios where players may take advantage of the situation and insert a bill or ticket, then cashout and move to the next EGM, but not place a bet, in an effort to purposefully disable machines throughout the casino floor.

In another embodiment, a player occupying event can be detected through the use of motion sensors coupled to the EGM or SMIB in accordance with the present disclosure. If motion is detected within range of the sensors, or a designated subset of sensor range, then a player occupying event can be generated. This may additionally require the player activity can be in the form of the player placing a minimum bet to avoid scenarios where players may take advantage of the situation and briefly sit in front of an EGM to purposefully disable a machine, and then get up and move to the next machine.

In another embodiment, a player occupying event can be detected through use of a camera (traditional or infrared) that detects if a player has occupied the EGM in accordance with the present disclosure. This can be detected based upon monitoring the complete field of view of the camera, or a configured sub-set of the field of view. This could optionally require the player activity can be in the form of the player additionally placing a minimum bet to avoid scenarios where players may take advantage of the situation and sit in front of an EGM to purposefully disable a machine, and then get up and move to the next machine.

### Detecting a Player Departing Event

A player departing event can occur upon a detection of any one or more suitable detectable actions by a player in accordance with the present disclosure.

In one embodiment, the EGM or SMIB can detect that a player has left an EGM (and that the EGM thus needs to be sanitized) through detection of the player removing their card from the card reader attached to the EGM or SMIB in accordance with the present disclosure. If the player initiated their session using a wireless mechanism (such as via Cardless Connect, etc.), then lack of a signal, or a signal strength below a configured threshold, or a disconnect of the wireless communications channel may also result in the triggering of a player departing event in accordance with the present disclosure.

In another embodiment, lack of player activity for a period of time (ex: 3 minutes) may result in detection of a player departing event as the player may have left the EGM without removing their player tracking card, or by properly closing the wireless communications channel between their mobile device and the EGM/SMIB in accordance with the present disclosure. Lack of player activity can be detected by the player not interacting with the EGM, by not playing a game, by not pressing the cashout button, by not triggering any activity that causes the EGM to generate an event or move machine meters, etc. The present disclosure contemplates that special care must be taken to not inappropriately generate a player departing event when the EGM is in a state where player activity is not expected. For example, if the player wins a jackpot, the EGM may lockup into a W2G jackpot handpay, which requires a casino staff member to come out to the EGM, give the player jurisdictionally appropriate tax forms (ex: US IRS W2-G tax form), and then finally the staff member will keyoff the jackpot to pay the player (by either crediting the jackpot amount to the credit meter or by handpaying the player with cash, ticket or other appropriate instrument). When the EGM is in a handpay, the timers evaluating inactivity can be paused when the handpay process starts and reset after the handpay process completes.

In another embodiment, the EGM or SMIB can be connected to motion sensors that can detect that a player who was sitting at the EGM has departed in accordance with the present disclosure. This can be detected by lack of player motion within a given time period.

In another embodiment, the EGM or SMIB can detect that a player has left the EGM by a player pressing the cashout button to have the EGM print a ticket or to transfer their funds on the credit meter to a cashless system in accordance with the present disclosure. The solution may trigger a departing player event immediately, or it may wait for up to a given timeout value, as the player at the EGM may not actually leave and may instead insert cash or additional funds into the EGM in order to start a new play session.

In another embodiment, the EGM or SMIB may generate a player departing event after the EGM's credit meter has gone to 0 (zero) in accordance with the present disclosure. The solution may trigger a departing player event immediately, or it may wait for up to a given timeout value, as the player at the EGM may insert cash or additional funds into the EGM in order to start a new play session. The present disclosure contemplates that special care must be taken to not inappropriately generate a player departing event when the EGM is in a state where player activity is not expected. For example, if the player wins a jackpot, the EGM may lockup into a W2G jackpot handpay, which requires a casino staff member to come out to the EGM, give the player jurisdictionally appropriate tax forms (ex: US IRS W2-G tax form), and then finally the staff member will keyoff the jackpot to pay the player (by either crediting the jackpot amount to the credit meter or by handpaying the player with cash, ticket or other appropriate instrument). When the EGM is in a handpay, the timers evaluating inactivity may be paused when the handpay process starts and reset after the handpay process completes.

In another embodiment, the EGM or SMIB may generate a player departing event responsive to termination event associated with game play. For example, if the gaming system or EGM no longer allows game play at a certain period of time, that that termination can be the player departing event. In one such example, if a lottery game has a cut off or playing at 9:30 pm for a 10:00 pm draw, reaching such cut off time can be a player departing event. In another such example, the end of the time for a tournament can be a player departing event. In certain such embodiments, if the EGM is disabled, the player will leave the EGM and thus that can be considered the player departing event.

In another embodiment, the EGM or SMIB can be connected to a video or infrared camera that can detect if a player is no longer in front of the EGM in accordance with the present disclosure. This camera can detect this by determining if a player is not located within a certain field of view or area covered by the camera. The solution may trigger a departing player event immediately, or it may wait for up to a given timeout value before generating a departing player event.

### Reacting to a Player Departing Event

After a Player Departing Event is detected, the EGM or SMIB may update its state to a state that describes that the cabinet of the EGM needs to be sanitized in accordance with the present disclosure. In one embodiment, the display of the EGM or SMIB may be updated to display a visual indicator that describes to both the player and operator that sanitization is required. This could include the rendering of a notification in the corner of the primary screen or secondary screen of the EGM, or the LCD or TFT display attached to the EGM. In another embodiment, the EGM's candle may light up, indicating that servicing is required. In another embodiment, the EGM may be disabled and the “EGM Disabled”, or “Out of Service” indication or tilt displayed on the main screen of the slot machine can indicate to a player that sanitization is required. If reacting to a player departing event is controlled by the EGM, then it may immediately disable the EGM or place it into an “out of service” mode. If reacting to a player departing event is the responsibility of the SMIB, then the SMIB can remotely disable the EGM using commands in various slot to host system protocols such as G2S, SAS, etc.

### Notifying the Resident CMS that the EGM Requires Sanitization

The EGM or SMIB may then communicate this state change to the connected host system so that a member of the casino staff can be dispatched to the EGM to perform the sanitization process in accordance with the present disclosure. The EGM or SMIB can notify the host system by publishing a message, or event to the host system to notify it that the EGM is awaiting sanitization or requires sanitization by a casino staff member. In one embodiment, the EGM could publish this event to the system using one of the commonly used EGM to host system protocols supported by EGMs, such as the SAS or G2S protocols. In another embodiment, the SMIB or EGM can publish this event using a protocol that's proprietary and not an industry standard, such as a message bus-based protocol or other protocol that leverages common network-based or serial-based communications technologies. In another embodiment, the SMIB or EGM can generate an existing event that describes a more general occurrence at the EGM that requires operator attention to resolve, such as the “change lamp” or EGM “service button” being pressed, which typically is used by the CMS to dispatch an attendant to the EGM to help a player resolve an issue.

Upon receipt of this event in the system, the resident system can use one or more methods to dispatch a staff member to the affected EGM to perform the sanitization process in accordance with the present disclosure. In one embodiment, a casino staff member may be sitting at a workstation and be responsible for manually dispatching a staff member to perform the sanitization process by notifying floor staff over a radio, cellular phone, or text message to perform the sanitization process at the identified EGM. In another embodiment, a casino staff member may have a mobile device that is notified automatically by the resident casino management system that sanitization is required at the identified EGM.

### Completing the Sanitization Process

Finally, after the casino staff member has completed the sanitization process, the casino staff member needs to change the state of the EGM or SMIB in order for the EGM to be safely available for play in accordance with the present disclosure.

In one example embodiment, the casino staff member may insert an employee, attendant, or 9s card into the card reader attached to the EGM or SMIB and they may be required to select an on-screen menu item to inform the EGM or SMIB that the sanitization process has been successfully completed.

In another example embodiment, the act of presenting the employee's ID (employee card, attendant card, 9s card) may be used by the EGM or SMIB to be informed that the sanitization process has been successfully completed. Other forms of employee ID may also be supported, such as NFC tags, passes in the employee's Apple Wallet or Google Wallet, initiation of a secure connection with the EGM or SMIB over Bluetooth Low Energy (BLE) using a casino employee mobile application, or use of other wireless technologies, etc.

In another example embodiment, which may be more appropriate for an EGM controlled sanitization workflow, the casino staff member may trigger the state change by turning the jackpot or attendant key of the EGM, opening the main door of the EGM, etc.

In another example embodiment, the casino staff member pay enter a PIN or passcode into the EGM or SMIB to record that the sanitization process has been completed.

In another example embodiment, a “sanitization reset ticket” can be inserted by the attendant to the EGM and the EGM, SMIB, or CMS could be pre-programmed to recognize the ticket presented as a “sanitization reset ticket”. For example, the EGM, SMIB, or CMS could be pre-programmed with the specific validation ID of the ticket presented to recognize it as a “sanitization reset ticket”. Alternatively, the EGM, SMIB, or CMS could recognize a “sanitization reset ticket” as any ticket whose validation ID contains a well-known or configured prefix value, such as “99”. After insertion of the “sanitization reset ticket” into the EGM's bill validator, the EGM, SMIB, or CMS may take actions to record the sanitization process has completed. Finally, the “sanitization reset ticket” may be rejected by the EGM and returned to the attendant by the bill validator so the ticket can be used at another EGM that requires sanitization.

In another example embodiment, the sanitization device itself could complete the process. For example, the EGM can be sanitized by an overhead UV light assembly. When that UV light assembly finishes (such as after 5 minutes of illuminating the surfaces of the EGM), the UV light assembly could send a signal notifying the casino management system that the EGM is sanitized and/or that the sanitization process is complete. In certain such embodiments, the UV light assembly could be in the form of a wand that the attendant uses and the wand notifies the casino management system that the sanitization process is complete.

When the EGM or SMIB is informed by the operator of the state change that needs to occur, the EGM or SMIB will then update or change the state of a visual indicator that is used to inform players if the EGM requires sanitization or if the EGM is safe to use in accordance with the present disclosure. In one embodiment, this may result in the change of a visual indicator that's displayed on the EGM. In another embodiment, this may result in the disablement of the EGM's candle (that was previously enabled and flashing to indicate that sanitization is required). In another embodiment, the EGM may be enabled or removed from the “out of service” state. If this workflow is controlled and managed by a SMIB, the change of the EGM's state can be triggered use of a commonly supported EGM to slot system protocol, such as SAS or G2S.

Once the EGM or SMIB has performed the state change, it may then, or concurrent with the state change, notify the resident CMS that the state change has occurred, which allows the resident system to record the state change for later inspection in accordance with the present disclosure. The EGM or SMIB may notify the resident CMS by publishing an event, or sending a message to the resident CMS over an existing EGM to casino slot system protocol, such as SAS, G2S, etc. Alternatively, the EGM or SMIB may notify the resident CMS by publishing the event using a proprietary protocol supported by the resident CMS, such as a message bus-based protocol, or protocol based upon common industry network communications technologies, etc.

It should be appreciated from the above that the present disclosure contemplates for various embodiments four different states that an EGM can be in. These states include: (1) a “clean” state in which the EGM is not being played and has been cleaned; (2) a “dirty” or “active” state where game play by a player has be detected at the EGM; (3) an “eligible to be locked” state where a player departing event has been detected and suitable timers or other conditions have expired; and (4) a “locked’ state where the EGM is locked and may display a suitable message, and also requires cleaning before being returned to the “clean” state.

In further alternative embodiments of the present disclosure, these above described processes can be employed for other functions associated with the EGMs beside sanitization. For example, if the casino desired to provide an object (such as fresh flowers) associated with an EGM for a new player that plays that EGM, the above processes can be used for cleaning and/or placing such object on or in association with the EGM. In various embodiments of the present disclosure, when the EGM is unavailable for play during the above described processes, the EGM and specifically a display device of the EGM may or may not display a suitable message regarding why the EGM is out of service during the period that the EGM is actually out of service. In certain embodiments, the EGM can provide a location in the casino of an identical or similar EGM for a player that approaches such out of service EGM.

In various embodiments of the present disclosure, after the EGM is cleaned and brought back into service such as in accordance with the above described processes, the EGM may or may not display a suitable message regarding the recent cleaning service. The message may be a generic message such as “THIS EGM WAS CLEANED AFTER THE LAST PLAYER LEFT” or a more specific message such as “THIS EGM WAS CLEANED 3 MINUTES AGO AFTER THE LAST PLAYER LEFT”. In various embodiments, the amount of time from the last cleaning may be maintained by the EGM and suitably tracked and displayed. It should be appreciated that other suitable sanitization indicators may be displayed by or used in conjunction with such EGMs in accordance with the present disclosure. In certain such embodiments, the candle of the EGM is also suitably used such as described herein.

In various embodiments of the present disclosure, groups of EGMs such as adjacent EGMs are taken out of service cleaning processes at the same time for efficiency purposes.

In various embodiments of the present disclosure, the state of the EGM and the processes described above take into account any bonus games that may be triggered in association with the EGM and play of such bonus games.

It should be appreciated from the above that various embodiments of the present disclosure provide a casino management system including a processor and a memory device that stores a plurality of instructions, which when executed by the processor, cause the processor to: (a) responsive to receiving a player departing event signal representing an indication that a player has left an EGM, cause a sanitization requirement notification associated with the EGM to be provided to a casino staff member; and (b) responsive to receiving a sanitization complete event signal representing an indication that the EGM has been sanitized, cause a state of the EGM to indicate that the EGM has been sanitized. In various such embodiments, the plurality of instructions, when executed by the processor, cause the processor to, responsive to receiving the player departing event signal, cause the EGM to display an indication that the EGM needs to be sanitized. In various such embodiments, the plurality of instructions, when executed by the processor, cause the processor to, responsive to receiving the player departing event signal, cause the EGM to be in a locked state. In various such embodiments, the plurality of instructions, when executed by the processor, cause the processor to, responsive to receiving the sanitization complete event signal, causing the EGM to be in an unlocked state. In various such embodiments, the plurality of instructions, when executed by the processor, cause the processor to, responsive to receiving the sanitization complete event signal, cause the EGM display a sanitized message. In various such embodiments, the plurality of instructions, when executed by the processor, cause the processor to, responsive to receiving the sanitization complete event signal, record that a sanitization process has been completed for the EGM. In various such embodiments, the plurality of instructions, when executed by the processor, cause the processor to, responsive to receiving a player occupying event signal representing that the EGM is being played by a player, record that a sanitization process will be need for the EGM.

It should further be appreciated from the above that various embodiments of the present disclosure provide an EGM including a display device; a processor; and a memory device that stores a plurality of instructions, which when executed by the processor, cause the processor to: (a) responsive to detecting that a player has left the EGM, send a player departing event signal representing an indication that the EGM needs to be sanitized; and (b) responsive to receiving an indication that the EGM has been sanitized, display, via the display device, an indication that the EGM has been sanitized.

In various such embodiments, the plurality of instructions, when executed by the processor, cause the processor to detect that a player has left the EGM based on an occurrence of a departing event comprising at least one of: a detection of removal of a player card from a card reader, a detection of a lack of a signal from a player wireless communication device, a detection of a signal strength below a threshold from a player wireless communication device, a detection of a disconnect signal from a player wireless communication device, a lack of player activity at the EGM for a period of time, a lack of a change to one or more player meters of the EGM for a period of time, a detection of a player departing the EGM for a period of time from a motion sensor, a detection of a player departing the EGM for a period of time from a camera, and a detection of activation of a cash-out button of the EGM.

In various such embodiments, the plurality of instructions, when executed by the processor, cause the processor to detect that the player has left the EGM based on an occurrence of the player departing event and a period of time after the occurrence of the player departing event. In various such embodiments, the plurality of instructions, when executed by the processor, cause the processor to detect that the player has left the EGM based on an occurrence of a player departing event and a period of time after the occurrence of the player departing event. In various such embodiments, the plurality of instructions, when executed by the processor, cause the processor to, responsive to detecting that the player has left the EGM, lock the EGM to prevent another player from playing the EGM while locked. In various such embodiments, the plurality of instructions, when executed by the processor, cause the processor to, responsive to detecting that the player has left the EGM, cause the display device to display an indication that the EGM needs to be sanitized. In various such embodiments, the plurality of instructions, when executed by the processor, cause the processor to, responsive to detecting that the player has left the EGM, sending the player departing event signal to a casino management system. In various such embodiments, the plurality of instructions, when executed by the processor, cause the processor to receive the indication that the EGM has been sanitized, based on an occurrence of at least one of: receipt of a designated casino staff member card by a card reader, receipt of a designated input by a casino staff member via a menu displayed by the display device, receipt of a designated wireless communication from a casino staff member communication device, an actuation of a key in the EGM, an input of a PIN or passcode into an input device of the EGM, and a receipt of a sanitization reset ticket. In various such embodiments, the plurality of instructions, when executed by the processor, cause the processor to receive the indication that the EGM has been sanitized, based on a receipt of a sanitization reset ticket and a rejection of the sanitization reset ticket. In various such embodiments, the plurality of instructions, when executed by the processor, cause the processor to, responsive to receiving the indication that the EGM has been sanitized, unlock the EGM to enable another player to play the EGM. In various such embodiments, the plurality of instructions, when executed by the processor, cause the processor to, responsive to receiving the indication that the EGM has been sanitized, send a sanitization event signal representing an indication that the EGM has been sanitized.

It should further be appreciated from the above that various embodiments of the present disclosure provide an EGM including a display device; a processor; and a memory device that stores a plurality of instructions, which when executed by the processor, cause the processor to: (a) responsive to an occurrence of a sanitization event, cause the EGM to be in a clean state in which the EGM is not being played and has been sanitized; (b) responsive to an occurrence of a player occupying event, cause the EGM to be in an active state; and (c) responsive to an occurrence of a departing event, cause the EGM to be eligible to be in a locked state, and then cause the EGM to be in a locked state that requires an occurrence of a sanitization event before the EGM returns to the clean state. In various such embodiments, the plurality of instructions, when executed by the processor, cause the processor to responsive to an occurrence of a departing event, cause the display device to display an indication that the EGM needs to be sanitized.

### Example Flowcharts

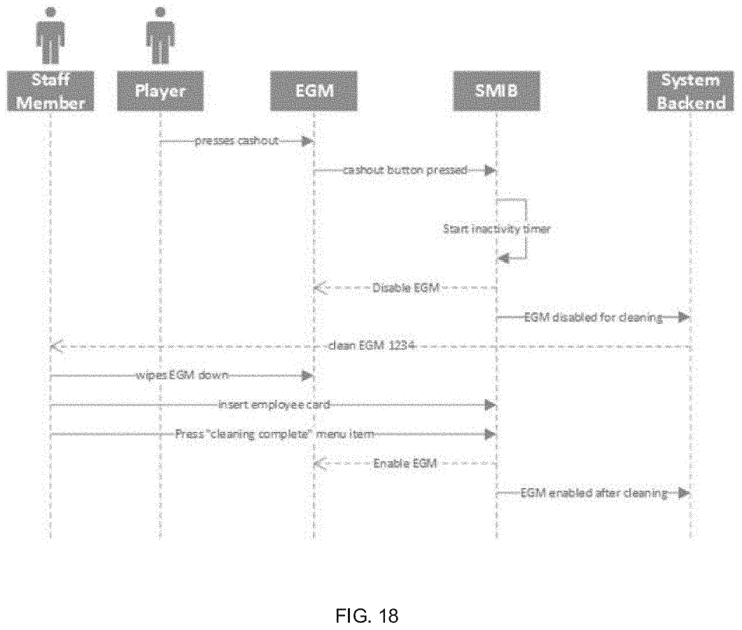

FIG. 18 depicts one example embodiment where the EGM is connected to a SMIB and the SMIB is communicating with a Casino Management System in accordance with the present disclosure.

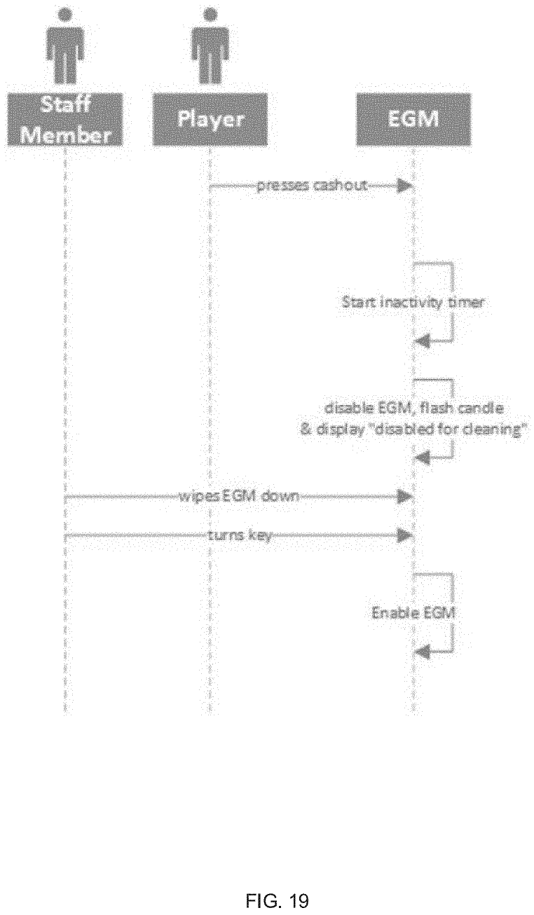

FIG. 19 describes a standalone implementation in the EGM itself that does not require the involvement of the resident Casino Management System in accordance with the present disclosure. The Casino Management System is notified of state changes, which it may be able to process and report to the operator, although it is asynchronous to the overall sanitization workflow implemented by the EGM.

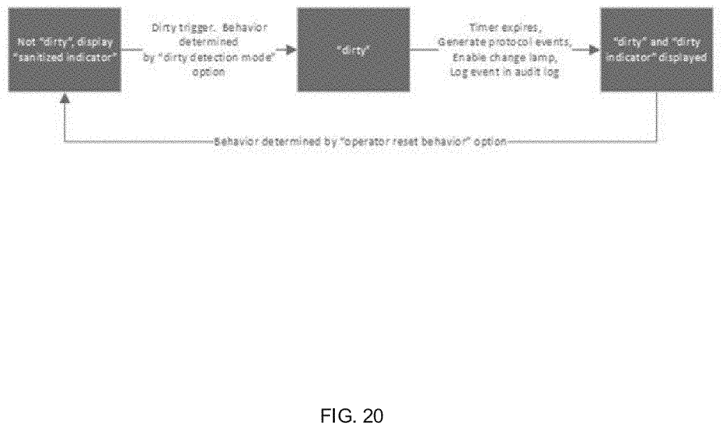

FIG. 20 depicts example states of an EGM during a sanitization workflow in accordance with example embodiments of the present disclosure. In various example embodiments, when the EGM is not in a “cleaning required” state, it may display the “clean indicator” so that players know the EGM is currently clean and is safe to use. The “clean indicator” may be displayed according to the value configured in the “clean indicator display mode” option. If the “clean indicator display mode” option is set to a value of “small”, which is the default value, then the “clean indicator” is represented by a small box of text displayed in the top right-hand corner of the screen. This area shares the screen with any door open icons and is effectively another style of door icon. If the “clean indicator display” option is set to a value of “large”, then a large overlay on the EGM's main monitor is displayed with a dismiss button.

In various example embodiments, upon detecting a “cleaning required trigger”, the EGM is in a “cleaning required” state and the “clean indicator” must be closed or no longer displayed. The EGM must support multiple “cleaning required” detection behaviors or “cleaning required triggers”, and the current behavior must be configured by the operator in the EGM's operator menus through the “cleaning required detection mode” option, which has the following possible values: (1) “require game play”—In this mode, the EGM only goes into a “cleaning required” state when a player places a wager; (2) “deposit of funds”—In this mode, the EGM only goes into a “cleaning required” state if the player inserts money into the EGM, such as inserting a bill or a voucher; (3) A host-initiated transfer (ex: SAS AFT) would also trigger the EGM to transition to a “cleaning required” state; and (4) “any activity”—Any activity on the EGM makes the EGM transition into a “cleaning required” state. This includes: touches on the touchscreen, presses of the button panel, and those methods covered by “deposit of funds” and “require game play”.

In various example embodiments, an EGM in the “cleaning required” state shall determine when to display a “cleaning required indicator” as follows: (1) 60 seconds (“Cleaning required inactivity timer at zero credits” configuration) after the credit meter goes to zero [Gives players time to add money after they have cashed out or the credit meter has hit zero]; and (2) If the credit meter is non-zero, 240 seconds (“Cleaning required inactivity timer with credits on machine” configuration) after the last game ended event [The longer time ensures that we don't lock uncarded players during a bonus game]. The timers shall be stopped when: (1) A handpay event is received (SAS Exception 51); reset and start the timers when the handpay is reset (SAS Exception 52); (2) On a game started event (SAS Exception 7E); reset and start the timers when the game ended event (SAS Exception 7F) is received; and (2) If any other player activity is detected, such as deposit of funds, press of a button on the button panel, touchscreen activity, etc. Reset and start the timers immediately thereafter.

In various example embodiments, when the EGM has determined it may display a “cleaning required indicator” (see definition below), the EGM may perform the following actions: (1) An event may be added to the EGM's event log to describe that a “cleaning required indicator” has been displayed and that the EGM needs to be cleaned; (2) Illuminate the candle change lamp; and (3) Generate SAS Exception 71 “Change lamp on” and G2S event G2S_CBE301 “Service Lamp On”.

In various example embodiments, the operator can reset the “cleaning required” state of the EGM by inserting a “cleaning required reset ticket” into the EGM. The EGM must process the validation ID of the inserted ticket before attempting to validate the ticket with the resident ticketing system. A “cleaning required reset ticket” must have the most significant 2 digits equal to the value configured in the “cleaning required reset ticket validation ID prefix” configuration item followed by a value of 0 in the 16 remaining digits. If the validation number of the inserted ticket matches the expected value for a “cleaning required reset ticket”, then the EGM must reset the “cleaning required” state of the EGM as described below and return the ticket to the operator. The EGM must not attempt to validate a valid “cleaning required reset ticket” with the resident ticketing system. Additionally, the “cleaning required indicator” may no longer be displayed. This can be achieved by no longer illuminating the change lamp of the candle. Generate SAS Exception 72 “Change lamp off” and G2S event G2S_CBE302 “Service Lamp Off”. An event may be added to the EGM's event log to describe that the EGM's “cleaning required” state has been reset and that the EGM has been sanitized. The EGM may display the “clean indicator” until the EGM detects that it has transitioned to a “cleaning required” state based upon the logic described by the current configuration value of the “cleaning required detection mode” option.

In various example embodiments, configuration options may be added to the operator menus, such as in a new sub-menu that has attendant level access in accordance with the present disclosure. These do not need to appear during cold boot: (1) “Enable/Disable cleaning workflow support” configuration option (Boolean, default false); (2) “Cleaning required inactivity timer at zero credits” configuration option (in seconds, default 60s); (3) “Cleaning required inactivity timer with credits on machine” configuration options (in seconds, default 240s); (4) “clean indicator display mode” configuration option, which has the following options: (a) “small”—The clean indicator will display in the door icon area of the EGM. This is the default value, and (b) “large”—The clean indicator will display large over the EGM's main monitor; (5) “cleaning required detection mode” configuration option, which has the following possible values: (a) “require game play”—see discussion above, (b) “deposit of funds”—see discussion above, (c) “any activity”—see discussion above; (5) “cleaning required reset ticket validation ID prefix”—This controls the 2-digit validation number prefix that must be used to determine the validation ID printed on a “cleaning required reset ticket”. This prefix is also used to validate a cleaning required reset ticket inserted by a casino staff member. The default value may be set to “88”, which controls the two most significant digits in the “cleaning required reset ticket” validation ID. The remaining 16 digits of the validation ID must be set to 0 and cannot be configured by an operator. The option must only support numbers between 01-79, and 81-88. The values 00, 80, and 89-99 cannot be set by an operator as those are reserved validation ID prefixes used by EZPay and other ticketing systems. (6) “cleaning required reset ticket title”—The ticket title to print on the “cleaning required reset ticket”. The default value may be “Cleaning Ticket”; (7) “Small clean indicator text value”—The text to display in the small “clean indicator” after the EGM's “cleaning required” state has been reset. The default value may be “Clean”. The system could require a certain number of maximum characters to limit screen real estate impact, such as 12 characters; and (8) “Large clean indicator text value”—The text to display in the large “clean indicator” after the EGM's “cleaning required” state has been reset. The default value may be “Cleaned for your safety, press any button to continue”. The system could require a certain number of maximum characters to limit screen real estate impact, such as 12 characters.

In various example embodiments, the configuration options page must also have a button to print a new “cleaning required reset ticket validation ID” based upon the value configured for “cleaning required reset ticket validation ID”. This may use a standard EZPay TITO ticket template and may use the existing property name, address line 1, address line 2 configured by the TITO system. The ticket title may be set to the value configured in “cleaning required reset ticket title” configuration option.

In various example embodiments, a large “cleaning indicator” is not a hard tilt and may not impact the Ascent Foundation in the same way a hard tilt does. A “clean indicator” is simply a notification to the operator or player that the EGM has been cleaned recently and has not been used since it was cleaned. Likewise, the “cleaning required indicator” is an internal state of the EGM where the candle is illuminated, but the EGM is not in a formal tilt state. In other words, a remote configuration or download host, such as Floor Manager, may still be able to put the EGM into remote configuration mode when a “cleaning required indicator” is displayed. Please note that an EGM cannot, per many jurisdictional requirements, be put into remote configuration mode if the EGM has an active “tilt”.

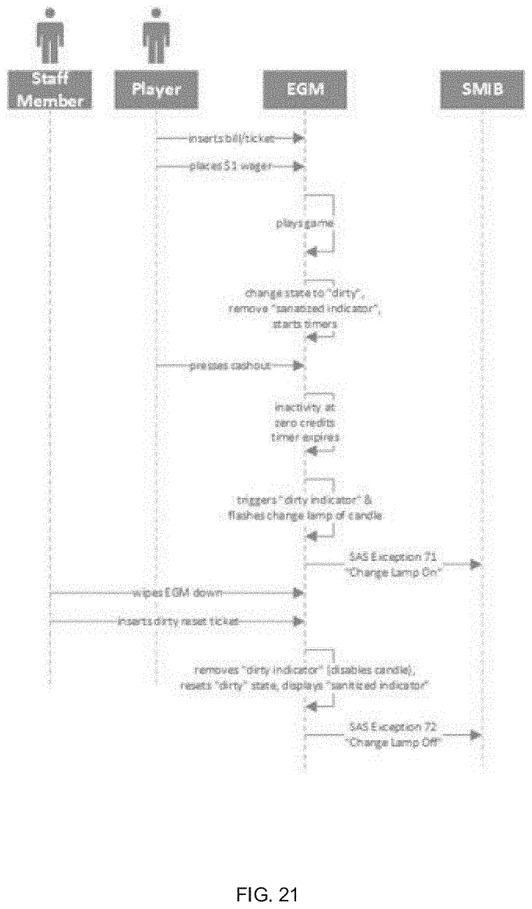

FIG. 21 depicts a flowchart of a player cashing into the EGM, playing a game, and then cashing out in accordance with the present disclosure. As the player interacts with the EGM, the EGM traverses the states accordingly as described above.

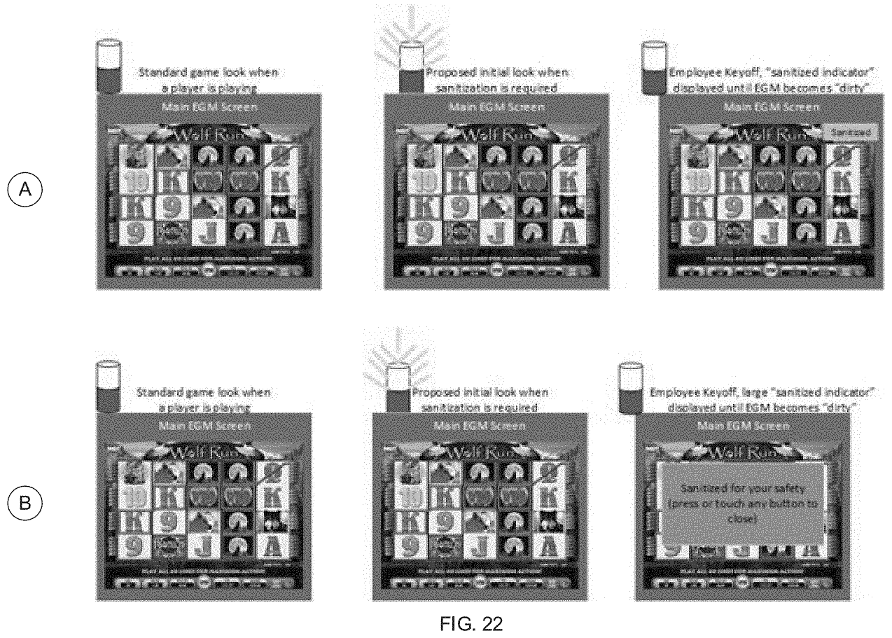

FIG. 22 depicts different lamp modes and user interface displays for an EGM which represent that current state of the EGM in accordance with the present disclosure. Players can easily tell which EGMs are safe to use, and which EGMs need to be sanitized. The change lamp of the candle flashes when the EGM is “dirty”. The “dirty” state can be reset by the player inserting a TITO-like ticket that has the validation number configured in the “dirty reset ticket validation ID”. After the “dirty” state is reset, the EGM will display a “sanitized indicator”. FIG. 15 depicts two possible look and feel of the EGM when it displays the “cleaning required indicator” (illumination of the candle), and after the operator clean the EGM by inserting a “cleaning required reset ticket”. Other user interfaces are possible in accordance with the present disclosure.

### Operation of Primary or Base Games and/or Secondary or Bonus Games

In various embodiments, an EGM may be implemented in one of a variety of different configurations. In various embodiments, the EGM may be implemented as one of: (a) a dedicated EGM in which computerized game programs executable by the EGM for controlling any primary or base games (referred to herein as “primary games”) and/or any secondary or bonus games or other functions (referred to herein as “secondary games”) displayed by the EGM are provided with the EGM before delivery to a gaming establishment or before being provided to a player; and (b) a changeable EGM in which computerized game programs executable by the EGM for controlling any primary games and/or secondary games displayed by the EGM are downloadable or otherwise transferred to the EGM through a data network or remote communication link; from a USB drive, flash memory card, or other suitable memory device; or in any other suitable manner after the EGM is physically located in a gaming establishment or after the EGM is provided to a player.

As generally explained above, in various embodiments in which the gaming system includes a central server, central controller, or remote host and a changeable EGM, the at least one memory device of the central server, central controller, or remote host stores different game programs and instructions executable by the at least one processor of the changeable EGM to control one or more primary games and/or secondary games displayed by the changeable EGM. More specifically, each such executable game program represents a different game or a different type of game that the at least one changeable EGM is configured to operate. In one example, certain of the game programs are executable by the changeable EGM to operate games having the same or substantially the same game play but different paytables. In different embodiments, each executable game program is associated with a primary game, a secondary game, or both. In certain embodiments, an executable game program is executable by the at least one processor of the at least one changeable EGM as a secondary game to be played simultaneously with a play of a primary game (which may be downloaded to or otherwise stored on the at least one changeable EGM), or vice versa.

In operation of such embodiments, the central server, central controller, or remote host is configured to communicate one or more of the stored executable game programs to the at least one processor of the changeable EGM. In different embodiments, a stored executable game program is communicated or delivered to the at least one processor of the changeable EGM by: (a) embedding the executable game program in a device or a component (such as a microchip to be inserted into the changeable EGM); (b) writing the executable game program onto a disc or other media; or (c) uploading or streaming the executable game program over a data network (such as a dedicated data network). After the executable game program is communicated from the central server, central controller, or remote host to the changeable EGM, the at least one processor of the changeable EGM executes the executable game program to enable the primary game and/or the secondary game associated with that executable game program to be played using the display device(s) and/or the input device(s) of the changeable EGM. That is, when an executable game program is communicated to the at least one processor of the changeable EGM, the at least one processor of the changeable EGM changes the game or the type of game that may be played using the changeable EGM.

In certain embodiments, the gaming system randomly determines any game outcome(s) (such as a win outcome) and/or award(s) (such as a quantity of credits to award for the win outcome) for a play of a primary game and/or a play of a secondary game based on probability data. In certain such embodiments, this random determination is provided through utilization of an RNG, such as a true RNG or a pseudo RNG, or any other suitable randomization process. In one such embodiment, each game outcome or award is associated with a probability, and the gaming system generates the game outcome(s) and/or the award(s) to be provided based on the associated probabilities. In these embodiments, since the gaming system generates game outcomes and/or awards randomly or based on one or more probability calculations, there is no certainty that the gaming system will ever provide any specific game outcome and/or award.

In certain embodiments, the gaming system maintains one or more predetermined pools or sets of predetermined game outcomes and/or awards. In certain such embodiments, upon generation or receipt of a game outcome and/or award request, the gaming system independently selects one of the predetermined game outcomes and/or awards from the one or more pools or sets. The gaming system flags or marks the selected game outcome and/or award as used. Once a game outcome or an award is flagged as used, it is prevented from further selection from its respective pool or set; that is, the gaming system does not select that game outcome or award upon another game outcome and/or award request. The gaming system provides the selected game outcome and/or award. Examples of this type of award evaluation are described in U.S. Pat. No. 7,470,183, entitled “Finite Pool Gaming Method and Apparatus”; U.S. Pat. No. 7,563,163, entitled “Gaming Device Including Outcome Pools for Providing Game Outcomes”; U.S. Pat. No. 7,833,092, entitled “Method and System for Compensating for Player Choice in a Game of Chance”; U.S. Pat. No. 8,070,579, entitled “Bingo System with Downloadable Common Patterns”; and U.S. Pat. No. 8,398,472, entitled “Central Determination Poker Game”.

In certain embodiments, the gaming system determines a predetermined game outcome and/or award based on the results of a bingo, keno, or lottery game. In certain such embodiments, the gaming system utilizes one or more bingo, keno, or lottery games to determine the predetermined game outcome and/or award provided for a primary game and/or a secondary game. The gaming system is provided or associated with a bingo card. Each bingo card consists of a matrix or array of elements, wherein each element is designated with separate indicia. After a bingo card is provided, the gaming system randomly selects or draws a plurality of the elements. As each element is selected, a determination is made as to whether the selected element is present on the bingo card. If the selected element is present on the bingo card, that selected element on the provided bingo card is marked or flagged. This process of selecting elements and marking any selected elements on the provided bingo cards continues until one or more predetermined patterns are marked on one or more of the provided bingo cards. After one or more predetermined patterns are marked on one or more of the provided bingo cards, game outcome and/or award is determined based, at least in part, on the selected elements on the provided bingo cards. Examples of this type of award determination are described in U.S. Pat. No. 7,753,774, entitled “Using Multiple Bingo Cards to Represent Multiple Slot Paylines and Other Class III Game Options”; U.S. Pat. No. 7,731,581, entitled “Multi-Player Bingo Game with Multiple Alternative Outcome Displays”; U.S. Pat. No. 7,955,170, entitled “Providing Non-Bingo Outcomes for a Bingo Game”; U.S. Pat. No. 8,070,579, entitled “Bingo System with Downloadable Common Patterns”; and U.S. Pat. No. 8,500,538, entitled “Bingo Gaming System and Method for Providing Multiple Outcomes from Single Bingo Pattern”.

In certain embodiments in which the gaming system includes a central server, central controller, or remote host and an EGM, the EGM is configured to communicate with the central server, central controller, or remote host for monitoring purposes only. In such embodiments, the EGM determines the game outcome(s) and/or award(s) to be provided in any of the manners described above, and the central server, central controller, or remote host monitors the activities and events occurring on the EGM. In one such embodiment, the gaming system includes a real-time or online accounting and gaming information system configured to communicate with the central server, central controller, or remote host. In this embodiment, the accounting and gaming information system includes: (a) a player database configured to store player profiles, (b) a player tracking module configured to track players (as described below), and (c) a credit system configured to provide automated transactions. Examples of such accounting systems are described in U.S. Pat. No. 6,913,534, entitled “Gaming Machine Having a Lottery Game and Capability for Integration with Gaming Device Accounting System and Player Tracking System,” and U.S. Pat. No. 8,597,116, entitled “Virtual Player Tracking and Related Services”.

As noted above, in various embodiments, the gaming system includes one or more executable game programs executable by at least one processor of the gaming system to provide one or more primary games and one or more secondary games. The primary game(s) and the secondary game(s) may comprise any suitable games and/or wagering games, such as, but not limited to: electro-mechanical or video slot or spinning reel type games; video card games such as video draw poker, multi-hand video draw poker, other video poker games, video blackjack games, and video baccarat games; video keno games; video bingo games; and video selection games.

In certain embodiments in which the primary game is a slot or spinning reel type game, the gaming system includes one or more reels in either an electromechanical form with mechanical rotating reels or in a video form with simulated reels and movement thereof. Each reel displays a plurality of indicia or symbols, such as bells, hearts, fruits, numbers, letters, bars, or other images that typically correspond to a theme associated with the gaming system. In certain such embodiments, the gaming system includes one or more paylines associated with the reels. The example EGM 100 shown in FIG. 1 includes a payline 152 and a plurality of reels 154. In certain embodiments, one or more of the reels are independent reels or unisymbol reels. In such embodiments, each independent reel generates and displays one symbol.

In various embodiments, one or more of the paylines is horizontal, vertical, circular, diagonal, angled, or any suitable combination thereof. In other embodiments, each of one or more of the paylines is associated with a plurality of adjacent symbol display areas on a requisite number of adjacent reels. In one such embodiment, one or more paylines are formed between at least two symbol display areas that are adjacent to each other by either sharing a common side or sharing a common corner (i.e., such paylines are connected paylines). The gaming system enables a wager to be placed on one or more of such paylines to activate such paylines. In other embodiments in which one or more paylines are formed between at least two adjacent symbol display areas, the gaming system enables a wager to be placed on a plurality of symbol display areas, which activates those symbol display areas.

In various embodiments, the gaming system provides one or more awards after a spin of the reels when specified types and/or configurations of the indicia or symbols on the reels occur on an active payline or otherwise occur in a winning pattern, occur on the requisite number of adjacent reels, and/or occur in a scatter pay arrangement.

In certain embodiments, the gaming system employs a ways to win award determination. In these embodiments, any outcome to be provided is determined based on a number of associated symbols that are generated in active symbol display areas on the requisite number of adjacent reels (i.e., not on paylines passing through any displayed winning symbol combinations). If a winning symbol combination is generated on the reels, one award for that occurrence of the generated winning symbol combination is provided. Examples of ways to win award determinations are described in U.S. Pat. No. 8,012,011, entitled “Gaming Device and Method Having Independent Reels and Multiple Ways of Winning”; U.S. Pat. No. 8,241,104, entitled “Gaming Device and Method Having Designated Rules for Determining Ways To Win”; and U.S. Pat. No. 8,430,739, entitled “Gaming System and Method Having Wager Dependent Different Symbol Evaluations”.

In various embodiments, the gaming system includes a progressive award. Typically, a progressive award includes an initial amount and an additional amount funded through a portion of each wager placed to initiate a play of a primary game. When one or more triggering events occurs, the gaming system provides at least a portion of the progressive award. After the gaming system provides the progressive award, an amount of the progressive award is reset to the initial amount and a portion of each subsequent wager is allocated to the next progressive award. Examples of progressive gaming systems are described in U.S. Pat. No. 7,585,223, entitled “Server Based Gaming System Having Multiple Progressive Awards”; U.S. Pat. No. 7,651,392, entitled “Gaming Device System Having Partial Progressive Payout”; U.S. Pat. No. 7,666,093, entitled “Gaming Method and Device Involving Progressive Wagers”; U.S. Pat. No. 7,780,523, entitled “Server Based Gaming System Having Multiple Progressive Awards”; and U.S. Pat. No. 8,337,298, entitled “Gaming Device Having Multiple Different Types of Progressive Awards”.

As generally noted above, in addition to providing winning credits or other awards for one or more plays of the primary game(s), in various embodiments the gaming system provides credits or other awards for one or more plays of one or more secondary games. The secondary game typically enables an award to be obtained addition to any award obtained through play of the primary game(s). The secondary game(s) typically produces a higher level of player excitement than the primary game(s) because the secondary game(s) provides a greater expectation of winning than the primary game(s) and is accompanied with more attractive or unusual features than the primary game(s). The secondary game(s) may be any type of suitable game, either similar to or completely different from the primary game.

In various embodiments, the gaming system automatically provides or initiates the secondary game upon the occurrence of a triggering event or the satisfaction of a qualifying condition. In other embodiments, the gaming system initiates the secondary game upon the occurrence of the triggering event or the satisfaction of the qualifying condition and upon receipt of an initiation input. In certain embodiments, the triggering event or qualifying condition is a selected outcome in the primary game(s) or a particular arrangement of one or more indicia on a display device for a play of the primary game(s), such as a “BONUS” symbol appearing on three adjacent reels along a payline following a spin of the reels for a play of the primary game. In other embodiments, the triggering event or qualifying condition occurs based on a certain amount of game play (such as number of games, number of credits, amount of time) being exceeded, or based on a specified number of points being earned during game play. Any suitable triggering event or qualifying condition or any suitable combination of a plurality of different triggering events or qualifying conditions may be employed.

In other embodiments, at least one processor of the gaming system randomly determines when to provide one or more plays of one or more secondary games. In one such embodiment, no apparent reason is provided for providing the secondary game. In this embodiment, qualifying for a secondary game is not triggered by the occurrence of an event in any primary game or based specifically on any of the plays of any primary game. That is, qualification is provided without any explanation or, alternatively, with a simple explanation. In another such embodiment, the gaming system determines qualification for a secondary game at least partially based on a game triggered or symbol triggered event, such as at least partially based on play of a primary game.

In various embodiments, after qualification for a secondary game has been determined, the secondary game participation may be enhanced through continued play on the primary game. Thus, in certain embodiments, for each secondary game qualifying event, such as a secondary game symbol, that is obtained, a given number of secondary game wagering points or credits is accumulated in a “secondary game meter” configured to accrue the secondary game wagering credits or entries toward eventual participation in the secondary game. In one such embodiment, the occurrence of multiple such secondary game qualifying events in the primary game results in an arithmetic or exponential increase in the number of secondary game wagering credits awarded. In another such embodiment, any extra secondary game wagering credits may be redeemed during the secondary game to extend play of the secondary game.

In certain embodiments, no separate entry fee or buy-in for the secondary game is required. That is, entry into the secondary game cannot be purchased; rather, in these embodiments entry must be won or earned through play of the primary game, thereby encouraging play of the primary game. In other embodiments, qualification for the secondary game is accomplished through a simple “buy-in.” For example, qualification through other specified activities is unsuccessful, payment of a fee or placement of an additional wager “buys-in” to the secondary game. In certain embodiments, a separate side wager must be placed on the secondary game or a wager of a designated amount must be placed on the primary game to enable qualification for the secondary game. In these embodiments, the secondary game triggering event must occur and the side wager (or designated primary game wager amount) must have been placed for the secondary game to trigger.

In various embodiments in which the gaming system includes a plurality of EGMs, the EGMs are configured to communicate with one another to provide a group gaming environment. In certain such embodiments, the EGMs enable players of those EGMs to work in conjunction with one another, such as by enabling the players to play together as a team or group, to win one or more awards. In other such embodiments, the EGMs enable players of those EGMs to compete against one another for one or more awards. In one such embodiment, the EGMs enable the players of those EGMs to participate in one or more gaming tournaments for one or more awards. Examples of group gaming systems are described in U.S. Pat. No. 8,070,583, entitled “Server Based Gaming System and Method for Selectively Providing One or More Different Tournaments”; U.S. Pat. No. 8,500,548, entitled “Gaming System and Method for Providing Team Progressive Awards”; and U.S. Pat. No. 8,562,423, entitled “Method and Apparatus for Rewarding Multiple Game Players for a Single Win”.

In various embodiments, the gaming system includes one or more player tracking systems. Such player tracking systems enable operators of the gaming system (such as casinos or other gaming establishments) to recognize the value of customer loyalty by identifying frequent customers and rewarding them for their patronage. Such a player tracking system is configured to track a player's gaming activity. In one such embodiment, the player tracking system does so through the use of player tracking cards. In this embodiment, a player is issued a player identification card that has an encoded player identification number that uniquely identifies the player. When the player's playing tracking card is inserted into a card reader of the gaming system to begin a gaming session, the card reader reads the player identification number off the player tracking card to identify the player. The gaming system timely tracks any suitable information or data relating to the identified player's gaming session. The gaming system also timely tracks when the player tracking card is removed to conclude play for that gaming session. In another embodiment, rather than requiring insertion of a player tracking card into the card reader, the gaming system utilizes one or more portable devices, such as a mobile phone, a radio frequency identification tag, or any other suitable wireless device, to track when a gaming session begins and ends. In another embodiment, the gaming system utilizes any suitable biometric technology or ticket technology to track when a gaming session begins and ends.

In such embodiments, during one or more gaming sessions, the gaming system tracks any suitable information or data, such as any amounts wagered, average wager amounts, and/or the time at which these wagers are placed. In different embodiments, for one or more players, the player tracking system includes the player's account number, the player's card number, the player's first name, the player's surname, the player's preferred name, the player's player tracking ranking, any promotion status associated with the player's player tracking card, the player's address, the player's birthday, the player's anniversary, the player's recent gaming sessions, or any other suitable data. In various embodiments, such tracked information and/or any suitable feature associated with the player tracking system is displayed on a player tracking display. In various embodiments, such tracked information and/or any suitable feature associated with the player tracking system is displayed via one or more service windows that are displayed on the central display device and/or the upper display device. Examples of player tracking systems are described in U.S. Pat. No. 6,722,985, entitled “Universal Player Tracking System”; U.S. Pat. No. 6,908,387, entitled “Player Tracking Communication Mechanisms in a Gaming Machine”; U.S. Pat. No. 7,311,605, entitled “Player Tracking Assembly for Complete Patron Tracking for Both Gaming and Non-Gaming Casino Activity”; U.S. Pat. No. 7,611,411, entitled “Player Tracking Instruments Having Multiple Communication Modes”; U.S. Pat. No. 7,617,151, entitled “Alternative Player Tracking Techniques”; and U.S. Pat. No. 8,057,298, entitled “Virtual Player Tracking and Related Services”.

### Web-Based Gaming

In various embodiments, the gaming system includes one or more servers configured to communicate with a personal gaming device—such as a smartphone, a tablet computer, a desktop computer, or a laptop computer—to enable web-based game play using the personal gaming device. In various embodiments, the player must first access a gaming website via an Internet browser of the personal gaming device or execute an application (commonly called an “app”) installed on the personal gaming device before the player can use the personal gaming device to participate in web-based game play. In certain embodiments, the one or more servers and the personal gaming device operate in a thin-client environment. In these embodiments, the personal gaming device receives inputs via one or more input devices (such as a touch screen and/or physical buttons), the personal gaming device sends the received inputs to the one or more servers, the one or more servers make various determinations based on the inputs and determine content to be displayed (such as a randomly determined game outcome and corresponding award), the one or more servers send the content to the personal gaming device, and the personal gaming device displays the content.

In certain such embodiments, the one or more servers must identify the player before enabling game play on the personal gaming device (or, in some embodiments, before enabling monetary wager-based game play on the personal gaming device). In these embodiments, the player must identify herself to the one or more servers, such as by inputting the player's unique playername and password combination, providing an input to a biometric sensor (e.g., a fingerprint sensor, a retinal sensor, a voice sensor, or a facial-recognition sensor), or providing any other suitable information.

Once identified, the one or more servers enable the player to establish an account balance from which the player can draw credits usable to wager on plays of a game. In certain embodiments, the one or more servers enable the player to initiate an electronic funds transfer to transfer funds from a bank account to the player's account balance. In other embodiments, the one or more servers enable the player to make a payment using the player's credit card, debit card, or other suitable device to add money to the player's account balance. In other embodiments, the one or more servers enable the player to add money to the player's account balance via a peer-to-peer type application, such as PayPal or Venmo. The one or more servers also enable the player to cash out the player's account balance (or part of it) in any suitable manner, such as via an electronic funds transfer, by initiating creation of a paper check that is mailed to the player, or by initiating printing of a voucher at a kiosk in a gaming establishment.

In certain embodiments, the one or more servers include a payment server that handles establishing and cashing out players' account balances and a separate game server configured to determine the outcome and any associated award for a play of a game. In these embodiments, the game server is configured to communicate with the personal gaming device and the payment device, and the personal gaming device and the payment device are not configured to directly communicate with one another. In these embodiments, when the game server receives data representing a request to start a play of a game at a desired wager, the game server sends data representing the desired wager to the payment server. The payment server determines whether the player's account balance can cover the desired wager (i.e., includes a monetary balance at least equal to the desired wager).

If the payment server determines that the player's account balance cannot cover the desired wager, the payment server notifies the game server, which then instructs the personal gaming device to display a suitable notification to the player that the player's account balance is too low to place the desired wager. If the payment server determines that the player's account balance can cover the desired wager, the payment server deducts the desired wager from the account balance and notifies the game server. The game server then determines an outcome and any associated award for the play of the game. The game server notifies the payment server of any nonzero award, and the payment server increases the player's account balance by the nonzero award. The game server sends data representing the outcome and any award to the personal gaming device, which displays the outcome and any award.

In certain embodiments, the one or more servers enable web-based game play using a personal gaming device only if the personal gaming device satisfies one or more jurisdictional requirements. In one embodiment, the one or more servers enable web-based game play using the personal gaming device only if the personal gaming device is located within a designated geographic area (such as within certain state or county lines or within the boundaries of a gaming establishment). In this embodiment, the geolocation module of the personal gaming device determines the location of the personal gaming device and sends the location to the one or more servers, which determine whether the personal gaming device is located within the designated geographic area. In various embodiments, the one or more servers enable non-monetary wager-based game play if the personal gaming device is located outside of the designated geographic area.

In various embodiments, the gaming system includes an EGM configured to communicate with a personal gaming device—such as a smartphone, a tablet computer, a desktop computer, or a laptop computer—to enable tethered mobile game play using the personal gaming device. Generally, in these embodiments, the EGM establishes communication with the personal gaming device and enables the player to play games on the EGM remotely via the personal gaming device. In certain embodiments, the gaming system includes a geo-fence system that enables tethered game play within a particular geographic area but not outside of that geographic area. Examples of tethering an EGM to a personal gaming device and geo-fencing are described in U.S. Patent Appl. Pub. No. 2013/0267324, entitled “Remote Gaming Method Allowing Temporary Inactivation Without Terminating Playing Session Due to Game Inactivity”.

### Social Network Integration

In certain embodiments, the gaming system is configured to communicate with a social network server that hosts or partially hosts a social networking website via a data network (such as the Internet) to integrate a player's gaming experience with the player's social networking account. This enables the gaming system to send certain information to the social network server that the social network server can use to create content (such as text, an image, and/or a video) and post it to the player's wall, newsfeed, or similar area of the social networking website accessible by the player's connections (and in certain cases the public) such that the player's connections can view that information. This also enables the gaming system to receive certain information from the social network server, such as the player's likes or dislikes or the player's list of connections. In certain embodiments, the gaming system enables the player to link the player's player account to the player's social networking account(s). This enables the gaming system to, once it identifies the player and initiates a gaming session (such as via the player logging in to a website (or an application) on the player's personal gaming device or via the player inserting the player's player tracking card into an EGM), link that gaming session to the player's social networking account(s). In other embodiments, the gaming system enables the player to link the player's social networking account(s) to individual gaming sessions when desired by providing the required login information.

For instance, in one embodiment, if a player wins a particular award (e.g., a progressive award or a jackpot award) or an award that exceeds a certain threshold (e.g., an award exceeding $1,000), the gaming system sends information about the award to the social network server to enable the server to create associated content (such as a screenshot of the outcome and associated award) and to post that content to the player's wall (or other suitable area) of the social networking website for the player's connections to see (and to entice them to play). In another embodiment, if a player joins a multiplayer game and there is another seat available, the gaming system sends that information to the social network sever to enable the server to create associated content (such as text indicating a vacancy for that particular game) and to post that content to the player's wall (or other suitable area) of the social networking website for the player's connections to see (and to entice them to fill the vacancy). In another embodiment, if the player consents, the gaming system sends advertisement information or offer information to the social network server to enable the social network server to create associated content (such as text or an image reflecting an advertisement and/or an offer) and to post that content to the player's wall (or other suitable area) of the social networking website for the player's connections to see. In another embodiment, the gaming system enables the player to recommend a game to the player's connections by posting a recommendation to the player's wall (or other suitable area) of the social networking website.

### Differentiating Certain Gaming Systems from General Purpose Computing Devices

Certain of the gaming systems described herein, such as EGMs located in a casino or another gaming establishment, include certain components and/or are configured to operate in certain manners that differentiate these systems from general purpose computing devices, i.e., certain personal gaming devices such as desktop computers and laptop computers.

For instance, EGMs are highly regulated to ensure fairness and, in many cases, EGMs are configured to award monetary awards up to multiple millions of dollars. To satisfy security and regulatory requirements in a gaming environment, hardware and/or software architectures are implemented in EGMs that differ significantly from those of general purpose computing devices. For purposes of illustration, a description of EGMs relative to general purpose computing devices and some examples of these additional (or different) hardware and/or software architectures found in EGMs are described below.

At first glance, one might think that adapting general purpose computing device technologies to the gaming industry and EGMs would be a simple proposition because both general purpose computing devices and EGMs employ processors that control a variety of devices. However, due to at least: (1) the regulatory requirements placed on EGMs, (2) the harsh environment in which EGMs operate, (3) security requirements, and (4) fault tolerance requirements, adapting general purpose computing device technologies to EGMs can be quite difficult. Further, techniques and methods for solving a problem in the general purpose computing device industry, such as device compatibility and connectivity issues, might not be adequate in the gaming industry. For instance, a fault or a weakness tolerated in a general purpose computing device, such as security holes in software or frequent crashes, is not tolerated in an EGM because in an EGM these faults can lead to a direct loss of funds from the EGM, such as stolen cash or loss of revenue when the EGM is not operating properly or when the random outcome determination is manipulated.

Certain differences between general purpose computing devices and EGMs are described below. A first difference between EGMs and general purpose computing devices is that EGMs are state-based systems. A state-based system stores and maintains its current state in a non-volatile memory such that, in the event of a power failure or other malfunction, the state-based system can return to that state when the power is restored or the malfunction is remedied. For instance, for a state-based EGM, if the EGM displays an award for a game of chance but the power to the EGM fails before the EGM provides the award to the player, the EGM stores the pre-power failure state in a non-volatile memory, returns to that state upon restoration of power, and provides the award to the player. This requirement affects the software and hardware design on EGMs. General purpose computing devices are not state-based machines, and a majority of data is usually lost when a malfunction occurs on a general purpose computing device.

A second difference between EGMs and general purpose computing devices is that, for regulatory purposes, the software on the EGM utilized to operate the EGM has been designed to be static and monolithic to prevent cheating by the operator of the EGM. For instance, one solution that has been employed in the gaming industry to prevent cheating and to satisfy regulatory requirements has been to manufacture an EGM that can use a proprietary processor running instructions to provide the game of chance from an EPROM or other form of non-volatile memory. The coding instructions on the EPROM are static (non-changeable) and must be approved by a gaming regulators in a particular jurisdiction and installed in the presence of a person representing the gaming jurisdiction. Any changes to any part of the software required to generate the game of chance, such as adding a new device driver used to operate a device during generation of the game of chance, can require burning a new EPROM approved by the gaming jurisdiction and reinstalling the new EPROM on the EGM in the presence of a gaming regulator. Regardless of whether the EPROM solution is used, to gain approval in most gaming jurisdictions, an EGM must demonstrate sufficient safeguards that prevent an operator or a player of an EGM from manipulating the EGM's hardware and software in a manner that gives him an unfair, and in some cases illegal, advantage.

A third difference between EGMs and general purpose computing devices is authentication—EGMs storing code are configured to authenticate the code to determine if the code is unaltered before executing the code. If the code has been altered, the EGM prevents the code from being executed. The code authentication requirements in the gaming industry affect both hardware and software designs on EGMs. Certain EGMs use hash functions to authenticate code. For instance, one EGM stores game program code, a hash function, and an authentication hash (which may be encrypted). Before executing the game program code, the EGM hashes the game program code using the hash function to obtain a result hash and compares the result hash to the authentication hash. If the result hash matches the authentication hash, the EGM determines that the game program code is valid and executes the game program code. If the result hash does not match the authentication hash, the EGM determines that the game program code has been altered (i.e., may have been tampered with) and prevents execution of the game program code. Examples of EGM code authentication are described in U.S. Pat. No. 6,962,530, entitled “Authentication in a Secure Computerized Gaming System”; U.S. Pat. No. 7,043,641, entitled “Encryption in a Secure Computerized Gaming System”; U.S. Pat. No. 7,201,662, entitled “Method and Apparatus for Software Authentication”; and U.S. Pat. No. 8,627,097, entitled “System and Method Enabling Parallel Processing of Hash Functions Using Authentication Checkpoint Hashes”.

A fourth difference between EGMs and general purpose computing devices is that EGMs have unique peripheral device requirements that differ from those of a general purpose computing device, such as peripheral device security requirements not usually addressed by general purpose computing devices. For instance, monetary devices, such as coin dispensers, bill validators, and ticket printers and computing devices that are used to govern the input and output of cash or other items having monetary value (such as tickets) to and from an EGM have security requirements that are not typically addressed in general purpose computing devices. Therefore, many general purpose computing device techniques and methods developed to facilitate device connectivity and device compatibility do not address the emphasis placed on security in the gaming industry.

To address some of the issues described above, a number of hardware/software components and architectures are utilized in EGMs that are not typically found in general purpose computing devices. These hardware/software components and architectures, as described below in more detail, include but are not limited to watchdog timers, voltage monitoring systems, state-based software architecture and supporting hardware, specialized communication interfaces, security monitoring, and trusted memory.

Certain EGMs use a watchdog timer to provide a software failure detection mechanism. In a normally-operating EGM, the operating software periodically accesses control registers in the watchdog timer subsystem to “re-trigger” the watchdog. Should the operating software fail to access the control registers within a preset timeframe, the watchdog timer will timeout and generate a system reset. Typical watchdog timer circuits include a loadable timeout counter register to enable the operating software to set the timeout interval within a certain range of time. A differentiating feature of some circuits is that the operating software cannot completely disable the function of the watchdog timer. In other words, the watchdog timer always functions from the time power is applied to the board.

Certain EGMs use several power supply voltages to operate portions of the computer circuitry. These can be generated in a central power supply or locally on the computer board. If any of these voltages falls out of the tolerance limits of the circuitry they power, unpredictable operation of the EGM may result. Though most modern general purpose computing devices include voltage monitoring circuitry, these types of circuits only report voltage status to the operating software. Out of tolerance voltages can cause software malfunction, creating a potential uncontrolled condition in the general purpose computing device. Certain EGMs have power supplies with relatively tighter voltage margins than that required by the operating circuitry. In addition, the voltage monitoring circuitry implemented in certain EGMs typically has two thresholds of control. The first threshold generates a software event that can be detected by the operating software and an error condition then generated. This threshold is triggered when a power supply voltage falls out of the tolerance range of the power supply, but is still within the operating range of the circuitry. The second threshold is set when a power supply voltage falls out of the operating tolerance of the circuitry. In this case, the circuitry generates a reset, halting operation of the EGM.

As described above, certain EGMs are state-based machines. Different functions of the game provided by the EGM (e.g., bet, play, result, points in the graphical presentation, etc.) may be defined as a state. When the EGM moves a game from one state to another, the EGM stores critical data regarding the game software in a custom non-volatile memory subsystem. This ensures that the player's wager and credits are preserved and to minimize potential disputes in the event of a malfunction on the EGM. In general, the EGM does not advance from a first state to a second state until critical information that enables the first state to be reconstructed has been stored. This feature enables the EGM to recover operation to the current state of play in the event of a malfunction, loss of power, etc. that occurred just before the malfunction. In at least one embodiment, the EGM is configured to store such critical information using atomic transactions.

Generally, an atomic operation in computer science refers to a set of operations that can be combined so that they appear to the rest of the system to be a single operation with only two possible outcomes: success or failure. As related to data storage, an atomic transaction may be characterized as series of database operations which either all occur, or all do not occur. A guarantee of atomicity prevents updates to the database occurring only partially, which can result in data corruption.

To ensure the success of atomic transactions relating to critical information to be stored in the EGM memory before a failure event (e.g., malfunction, loss of power, etc.), memory that includes one or more of the following criteria be used: direct memory access capability; data read/write capability which meets or exceeds minimum read/write access characteristics (such as at least 5.08 Mbytes/sec (Read) and/or at least 38.0 Mbytes/sec (Write)). Memory devices that meet or exceed the above criteria may be referred to as “fault-tolerant” memory devices.

Typically, battery-backed RAM devices may be configured to function as fault-tolerant devices according to the above criteria, whereas flash RAM and/or disk drive memory are typically not configurable to function as fault-tolerant devices according to the above criteria. Accordingly, battery-backed RAM devices are typically used to preserve EGM critical data, although other types of non-volatile memory devices may be employed. These memory devices are typically not used in typical general purpose computing devices.

Thus, in at least one embodiment, the EGM is configured to store critical information in fault-tolerant memory (e.g., battery-backed RAM devices) using atomic transactions. Further, in at least one embodiment, the fault-tolerant memory is able to successfully complete all desired atomic transactions (e.g., relating to the storage of EGM critical information) within a time period of 200 milliseconds or less. In at least one embodiment, the time period of 200 milliseconds represents a maximum amount of time for which sufficient power may be available to the various EGM components after a power outage event has occurred at the EGM.

As described previously, the EGM may not advance from a first state to a second state until critical information that enables the first state to be reconstructed has been atomically stored. After the state of the EGM is restored during the play of a game of chance, game play may resume and the game may be completed in a manner that is no different than if the malfunction had not occurred. Thus, for example, when a malfunction occurs during a game of chance, the EGM may be restored to a state in the game of chance just before when the malfunction occurred. The restored state may include metering information and graphical information that was displayed on the EGM in the state before the malfunction. For example, when the malfunction occurs during the play of a card game after the cards have been dealt, the EGM may be restored with the cards that were previously displayed as part of the card game. As another example, a bonus game may be triggered during the play of a game of chance in which a player is required to make a number of selections on a video display screen. When a malfunction has occurred after the player has made one or more selections, the EGM may be restored to a state that shows the graphical presentation just before the malfunction including an indication of selections that have already been made by the player. In general, the EGM may be restored to any state in a plurality of states that occur in the game of chance that occurs while the game of chance is played or to states that occur between the play of a game of chance.

Game history information regarding previous games played such as an amount wagered, the outcome of the game, and the like may also be stored in a non-volatile memory device. The information stored in the non-volatile memory may be detailed enough to reconstruct a portion of the graphical presentation that was previously presented on the EGM and the state of the EGM (e.g., credits) at the time the game of chance was played. The game history information may be utilized in the event of a dispute. For example, a player may decide that in a previous game of chance that they did not receive credit for an award that they believed they won. The game history information may be used to reconstruct the state of the EGM before, during, and/or after the disputed game to demonstrate whether the player was correct or not in the player's assertion. Examples of a state-based EGM, recovery from malfunctions, and game history are described in U.S. Pat. No. 6,804,763, entitled “High Performance Battery Backed RAM Interface”; U.S. Pat. No. 6,863,608, entitled “Frame Capture of Actual Game Play”; U.S. Pat. No. 7,111,141, entitled “Dynamic NV-RAM”; and U.S. Pat. No. 7,384,339, entitled, “Frame Capture of Actual Game Play”.

Another feature of EGMs is that they often include unique interfaces, including serial interfaces, to connect to specific subsystems internal and external to the EGM. The serial devices may have electrical interface requirements that differ from the “standard” EIA serial interfaces provided by general purpose computing devices. These interfaces may include, for example, Fiber Optic Serial, optically coupled serial interfaces, current loop style serial interfaces, etc. In addition, to conserve serial interfaces internally in the EGM, serial devices may be connected in a shared, daisy-chain fashion in which multiple peripheral devices are connected to a single serial channel.

The serial interfaces may be used to transmit information using communication protocols that are unique to the gaming industry. For example, IGT's Netplex is a proprietary communication protocol used for serial communication between EGMs. As another example, SAS is a communication protocol used to transmit information, such as metering information, from an EGM to a remote device. Often SAS is used in conjunction with a player tracking system.

Certain EGMs may alternatively be treated as peripheral devices to a casino communication controller and connected in a shared daisy chain fashion to a single serial interface. In both cases, the peripheral devices are assigned device addresses. If so, the serial controller circuitry must implement a method to generate or detect unique device addresses. General purpose computing device serial ports are not able to do this.

Security monitoring circuits detect intrusion into an EGM by monitoring security switches attached to access doors in the EGM cabinet. Access violations result in suspension of game play and can trigger additional security operations to preserve the current state of game play. These circuits also function when power is off by use of a battery backup. In power-off operation, these circuits continue to monitor the access doors of the EGM. When power is restored, the EGM can determine whether any security violations occurred while power was off, e.g., via software for reading status registers. This can trigger event log entries and further data authentication operations by the EGM software.

Trusted memory devices and/or trusted memory sources are included in an EGM to ensure the authenticity of the software that may be stored on less secure memory subsystems, such as mass storage devices. Trusted memory devices and controlling circuitry are typically designed to not enable modification of the code and data stored in the memory device while the memory device is installed in the EGM. The code and data stored in these devices may include authentication algorithms, random number generators, authentication keys, operating system kernels, etc. The purpose of these trusted memory devices is to provide gaming regulatory authorities a root trusted authority within the computing environment of the EGM that can be tracked and verified as original. This may be accomplished via removal of the trusted memory device from the EGM computer and verification of the secure memory device contents is a separate third party verification device. Once the trusted memory device is verified as authentic, and based on the approval of the verification algorithms included in the trusted device, the EGM is enabled to verify the authenticity of additional code and data that may be located in the gaming computer assembly, such as code and data stored on hard disk drives. Examples of trusted memory devices are described in U.S. Pat. No. 6,685,567, entitled “Process Verification”.

In at least one embodiment, at least a portion of the trusted memory devices/sources may correspond to memory that cannot easily be altered (e.g., “unalterable memory”) such as EPROMS, PROMS, Bios, Extended Bios, and/or other memory sources that are able to be configured, verified, and/or authenticated (e.g., for authenticity) in a secure and controlled manner.

According to one embodiment, when a trusted information source is in communication with a remote device via a network, the remote device may employ a verification scheme to verify the identity of the trusted information source. For example, the trusted information source and the remote device may exchange information using public and private encryption keys to verify each other's identities. In another embodiment, the remote device and the trusted information source may engage in methods using zero knowledge proofs to authenticate each of their respective identities.

EGMs storing trusted information may utilize apparatuses or methods to detect and prevent tampering. For instance, trusted information stored in a trusted memory device may be encrypted to prevent its misuse. In addition, the trusted memory device may be secured behind a locked door. Further, one or more sensors may be coupled to the memory device to detect tampering with the memory device and provide some record of the tampering. In yet another example, the memory device storing trusted information might be designed to detect tampering attempts and clear or erase itself when an attempt at tampering has been detected. Examples of trusted memory devices/sources are described in U.S. Pat. No. 7,515,718, entitled “Secured Virtual Network in a Gaming Environment”.

Mass storage devices used in a general purpose computing devices typically enable code and data to be read from and written to the mass storage device. In a gaming environment, modification of the gaming code stored on a mass storage device is strictly controlled and would only be enabled under specific maintenance type events with electronic and physical enablers required. Though this level of security could be provided by software, EGMs that include mass storage devices include hardware level mass storage data protection circuitry that operates at the circuit level to monitor attempts to modify data on the mass storage device and will generate both software and hardware error triggers should a data modification be attempted without the proper electronic and physical enablers being present. Examples of using a mass storage device are described in U.S. Pat. No. 6,149,522, entitled “Method of Authenticating Game Data Sets in an Electronic Casino Gaming System”.

Various changes and modifications to the present embodiments described herein will be apparent to those skilled in the art. Such changes and modifications can be made without departing from the spirit and scope of the present subject matter and without diminishing its intended technical scope. It is therefore intended that such changes and modifications be covered by the appended claims.

What is claimed is:

1. A casino management system comprising:
a processor; and
a memory device that stores a plurality of instructions, which when executed by the processor, cause the processor to:
responsive to receiving a player departing event signal representing an indication that a player has left an electronic gaming machine, cause a sanitization requirement notification associated with the electronic gaming machine to be provided to a casino staff member; and
responsive to receiving a sanitization complete event signal representing an indication that the electronic gaming machine has been sanitized, cause a state of the electronic gaming machine to indicate that the electronic gaming machine has been sanitized.

2. The casino management system of claim 1, wherein the plurality of instructions, when executed by the processor, cause the processor to, responsive to receiving the player departing event signal, cause the electronic gaming machine to display an indication that the electronic gaming machine needs to be sanitized.

3. The casino management system of claim 1, wherein the plurality of instructions, when executed by the processor, cause the processor to, responsive to receiving the player departing event signal, cause the electronic gaming machine to be in a locked state.

4. The casino management system of claim 3, wherein the plurality of instructions, when executed by the processor, cause the processor to, responsive to receiving the sanitization complete event signal, cause the electronic gaming machine to be in an unlocked state.

5. The casino management system of claim 3, wherein the plurality of instructions, when executed by the processor, cause the processor to, responsive to receiving the sanitization complete event signal, cause the electronic gaming machine to display a sanitized message.

6. The casino management system of claim 1, wherein the plurality of instructions, when executed by the processor, cause the processor to, responsive to receiving the sanitization complete event signal, record that a sanitization process has been completed for the electronic gaming machine.

7. The casino management system of claim 1, wherein the plurality of instructions, when executed by the processor, cause the processor to, responsive to receiving a player occupying event signal representing that the electronic gaming machine is being played by a player, record that a sanitization process will be needed for the electronic gaming machine.

8. An electronic gaming machine comprising:
a display device;
a processor; and
a memory device that stores a plurality of instructions, which when executed by the processor, cause the processor to:
responsive to detecting that a player has left the electronic gaming machine, send a player departing event signal representing an indication that the electronic gaming machine needs to be sanitized; and
responsive to receiving an indication that the electronic gaming machine has been sanitized, display, via the display device, an indication that the electronic gaming machine has been sanitized.

9. The electronic gaming machine of claim 8, wherein the plurality of instructions, when executed by the processor, cause the processor to detect that a player has left the electronic gaming machine based on an occurrence of a departing event comprising at least one of: a detection of removal of a player card from a card reader, a detection of a lack of a signal from a player wireless communication device, a detection of a signal strength below a threshold from a player wireless communication device, a detection of a disconnect signal from a player wireless communication device, a lack of player activity at the electronic gaming machine for a period of time, a lack of a change to one or more player meters of the electronic gaming machine for a period of time, a detection of a player departing the electronic gaming machine for a period of time from a motion sensor, a detection of a player departing the electronic gaming machine for a period of time from a camera, and a detection of activation of a cash-out button of the electronic gaming machine.

10. The electronic gaming machine of claim 9, wherein the plurality of instructions, when executed by the processor, cause the processor to detect that the player has left the electronic gaming machine based on an occurrence of the player departing event and a period of time after the occurrence of the player departing event.

11. The electronic gaming machine of claim 8, wherein the plurality of instructions, when executed by the processor, cause the processor to detect that the player has left the electronic gaming machine based on an occurrence of a player departing event and a period of time after the occurrence of the player departing event.

12. The electronic gaming machine of claim 8, wherein the plurality of instructions, when executed by the processor, cause the processor to, responsive to detecting that the player has left the electronic gaming machine, lock the electronic gaming machine to prevent another player from playing the electronic gaming machine while locked.

13. The electronic gaming machine of claim 8, wherein the plurality of instructions, when executed by the processor, cause the processor to, responsive to detecting that the player has left the electronic gaming machine, cause the display device to display an indication that the electronic gaming machine needs to be sanitized.

14. The electronic gaming machine of claim 8, wherein the plurality of instructions, when executed by the processor, cause the processor to, responsive to detecting that the player has left the electronic gaming machine, send the player departing event signal to a casino management system.

15. The electronic gaming machine of claim 8, wherein the plurality of instructions, when executed by the processor, cause the processor to receive the indication that the electronic gaming machine has been sanitized, based on an occurrence of at least one of: receipt of a designated casino staff member card by a card reader, receipt of a designated input by a casino staff member via a menu displayed by the display device, receipt of a designated wireless communication from a casino staff member communication device, an actuation of a key in the electronic gaming machine, an input of a PIN or passcode into an input device of the electronic gaming machine, and a receipt of a sanitization reset ticket.

16. The electronic gaming machine of claim 8, wherein the plurality of instructions, when executed by the processor, cause the processor to receive the indication that the electronic gaming machine has been sanitized, based on a receipt of a sanitization reset ticket and a rejection of the sanitization reset ticket.

17. The electronic gaming machine of claim 8, wherein the plurality of instructions, when executed by the processor, cause the processor to, responsive to receiving the indication that the electronic gaming machine has been sanitized, unlock the electronic gaming machine to enable another player to play the electronic gaming machine.

18. The electronic gaming machine of claim 8, wherein the plurality of instructions, when executed by the processor, cause the processor to, responsive to receiving the indication that the electronic gaming machine has been sanitized, send a sanitization event signal representing an indication that the electronic gaming machine has been sanitized.

19. An electronic gaming machine comprising:
a display device;
a processor; and
a memory device that stores a plurality of instructions, which when executed by the processor, cause the processor to:
responsive to an occurrence of a sanitization event, cause the electronic gaming machine to be in a clean state in which the electronic gaming machine is not being played and has been sanitized;
responsive to an occurrence of a player occupying event, cause the electronic gaming machine to be in an active state; and
responsive to an occurrence of a departing event, cause the electronic gaming machine to be eligible to be in a locked state, and then cause the electronic gaming machine to be in a locked state that requires an occurrence of a sanitization event before the electronic gaming machine returns to the clean state.

20. The electronic gaming machine of claim 19, wherein the plurality of instructions, when executed by the processor, cause the processor to responsive to an occurrence of a departing event, cause the display device to display an indication that the electronic gaming machine needs to be sanitized.
